(19) United States
(12) Patent Application Publication
(10) Pub. No.: US 2024/0408082 A1
(43) Pub. Date: Dec. 12, 2024

(54) METHODS FOR TREATING CANCERS AND TUMORS

(71) Applicant: BEYONDSPRING PHARMACEUTICALS, INC., Florham Park, NJ (US)

(72) Inventors: George Kenneth Lloyd, Florham Park, NJ (US); Ramon Mohanlal, Florham Park, NJ (US); James R. Tonra, Florham Park, NJ (US); Lan Huang, Florham Park, NJ (US)

(21) Appl. No.: 18/698,677

(22) PCT Filed: Oct. 6, 2022
(86) PCT No.: PCT/US2022/077702

§ 371 Date: Apr. 4, 2024

Related U.S. Application Data
(60) Provisional application No. 63/253,332, filed on Oct. 7, 2021.

(51) Int. Cl. A61K 31/496; A61P 35/00

(52) U.S. Cl. CPC A61K 31/496; A61P 35/00

(57) ABSTRACT

Disclosed herein are methods of treating, preventing or ameliorating a disease or condition associated with cancer or a tumor. Some embodiments relate to methods of inhibiting proliferation of a cancer cell. Some embodiments relate to a method of inducing apoptosis in a cancer cell. In some embodiments, the methods include administering a compound of Formula (I) to a subject in need thereof.

### CROSS REFERENCE TO RELATED APPLICATIONS

This patent application claims the benefit of priority to U.S. Provisional Application No. 63/253,332, filed Oct. 7, 2021. All of the foregoing applications are fully incorporated herein by reference in their entireties for all purposes.

### FIELD OF THE DISCLOSURE

The present disclosure relates to the field of chemistry and medicine. More particularly, the present disclosure relates to compositions containing Plinabulin, and its use in treatment.

### BACKGROUND

Gastric cancer is a disease in which malignant cells form in the lining of the stomach. Stomach or gastric cancer can develop in any part of the stomach and may spread throughout the stomach and to other organs, particularly the esophagus, lungs, and liver. Stomach cancer is the fourth most common cancer worldwide, with 930,000 cases diagnosed in 2002. In addition, it is a disease with a high death rate (˜800,000 per year), making it the second most common cause of cancer death worldwide after lung cancer.

Small cell lung cancer (SCLC) is named according to the size of the cancer cells when observed under a microscope and has to be differentiated from non-small cell lung cancer (NSCLC). SCLC accounts for about 10% to 15% of all lung cancers (American Cancer Society, 2015a).

Both lung cancers (SCLC and NSCLC) are the second most common cancer in both men and women. Lung cancer is the leading cause of cancer death, which accounts for about 25%. Thus, more people die of lung cancer than colon, breast, and prostate cancers combined yearly. Furthermore, lung cancers account for about 13% (more than 1.8 million) of all new cancers. Lung cancer mainly occurs in older people. The average age at the time of diagnosis is about 70. Fewer than 2% of all cases are diagnosed in people younger than 45. The treatment and prognosis of SCLC depend strongly on the diagnosed cancer stage. The staging of SCLC based on clinical results is more common than pathologic staging. The clinical staging uses the results of the physical examination, various imaging tests, and biopsies. According to the data introduced by the American Cancer Society, the 5-year relative survival rate accounts to 31% for stage I, 19% for stage II, 8% for stage III, and 2% for stage IV.

Treatment of patients with triple-negative breast cancer (TNBC), lacking estrogen receptor (ER) and progesterone receptor (PR) expression as well as human epidermal growth factor receptor 2 (HER2) amplification, has been challenging due to the heterogeneity of the disease and the absence of well-defined molecular targets (Pegram M D, et al. J Clin Oncol. 1998; 16(8):2659-2671; Wiggans R G, et al. Cancer Chemother Pharmacol. 1979; 3(1):45-48; Carey L A, et al. Clin Cancer Res. 2007; 13(8):2329-2334). TNBCs constitute 10%-20% of all breast cancers, more frequently affect younger patients, and are more prevalent in African-American women (Morris G J, et al. Cancer. 2007; 110(4):876-884). TNBC tumors are generally larger in size, are of higher grade, have lymph node involvement at diagnosis, and are biologically more aggressive (Haffty B G, et al. J Clin Oncol. 2006; 24(36):5652-5657). Despite having higher rates of clinical response to presurgical (neoadjuvant) chemotherapy, TNBC patients have a higher rate of distant recurrence and a poorer prognosis than women with other breast cancer subtypes (Haffty B G, et al. J Clin Oncol. 2006; 24(36):5652-5657; Dent R, et al. Clin Cancer Res. 2007; 13 (15 pt 1):4429-4434). Less than 30% of women with metastatic TNBC survive 5 years, and almost all die of their disease despite adjuvant chemotherapy, which is the mainstay of treatment (Dent R, et al. Clin Cancer Res. 2007; 13 (15 pt 1):4429-4434).

As such, there remains a need to develop effective treatments for these and other aggressive types of cancers.

### SUMMARY OF THE DISCLOSURE

Some aspects relate to a method of treating a cancer in a subject in need thereof. In some embodiments, the method includes administering to the subject a compound of Formula (I) as a monotherapy,

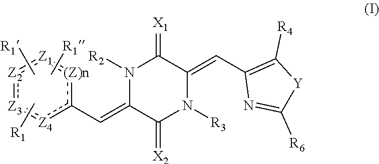

or a pharmaceutically acceptable salt thereof, wherein:
R₁, R₄, and R₆, are each separately selected from the group consisting of a hydrogen atom, a deuterium atom, a halogen atom, and saturated C₁,—C₂₄ alkyl, unsaturated C₁-C₂₄ alkenyl, cycloalkyl, cycloalkenyl, alkoxy, cycloalkoxy, aryl, substituted aryl, heteroaryl, substituted heteroaryl, amino, substituted amino, nitro, azido, substituted nitro, phenyl, and substituted phenyl groups, hydroxy, carboxy, —CO—O—R₇, cyano, alkylthio, halogenated alkyl including polyhalogenated alkyl, halogenated carbonyl, and carbonyl —CH₂CO—R₇, wherein R₇ is selected from a hydrogen atom, a halogen atom, and saturated C₁-C₂₄ alkyl, unsaturated C₁-C₂₄ alkenyl, cycloalkyl, cycloalkenyl, alkoxy, cycloalkoxy, aryl, substituted aryl, heteroaryl, substituted heteroaryl, amino, substituted amino, nitro, azido, substituted nitro, phenyl, and substituted phenyl groups;
R₁′ and R₁″ are each independently selected from the group consisting of a hydrogen atom, a deuterium atom, a halogen atom, and saturated C₁-C₂₄ alkyl, unsaturated C₁-C₂₄ alkenyl, cycloalkyl, cycloalkenyl, alkoxy, cycloalkoxy, aryl, substituted aryl, heteroaryl, substituted heteroaryl, amino, substituted amino, nitro, azido, substituted nitro, phenyl, and substituted phenyl groups, hydroxy, carboxy, —CO—O—R₇, cyano, alkylthio, halogenated alkyl including polyhalogenated alkyl, halogenated carbonyl, and carbonyl —CH₂CO—R₇, wherein R₇ is selected from a hydrogen atom, a halogen atom, and saturated C₁-C₂₄ alkyl, unsaturated C₁-C₂₄ alkenyl, cycloalkyl, cycloalkenyl, alkoxy, cycloalkoxy, aryl, substituted aryl, heteroaryl, substituted heteroaryl, amino, substituted amino, nitro, azido, substituted nitro, phenyl, and substituted phenyl groups;
R, R₁′ and R₁″ are either covalently bound to one another or are not covalently bound to one another;
R₂, R₃, and R₅ are each separately selected from the group consisting of a hydrogen atom, a deuterium atom, a halogen atom, and saturated C₁-C₁₂ alkyl, unsaturated C₁-C₁₂ alkenyl, acyl, cycloalkyl, alkoxy, cycloalkoxy, aryl, substituted aryl, heteroaryl, substituted heteroaryl, amino, substituted amino, nitro, and substituted nitro groups, sulfonyl and substituted sulfonyl groups;
m is an integer equal to zero, one or two;
X₁ and X₂ are separately selected from the group consisting of an oxygen atom, a nitrogen atom and a sulfur atom, and
Y is selected from the group consisting of a NRs, an oxygen atom, a sulfur atom, a oxidized sulfur atom, a methylene group and a substituted methylene group;
Z, for each separate n, if non-zero, and Z₁, Z₂, Z₃ and Z₄ are each separately selected from a carbon atom, a sulfur atom, a nitrogen atom or an oxygen atom; and
the dashed bonds may be either single or double bonds,
wherein the cancer is selected from gastric cancer, small cell lung cancer, osteosarcoma, bladder cancer, and triple negative breast cancer.

In some embodiments, the cancer is gastric cancer. In some embodiments, the cancer is small cell lung cancer. In some embodiments, the cancer is triple negative breast cancer. In some embodiments, the compound of Formula (I) is administered at a dose from about 5 mg/m² to 150 mg/m². In some embodiments, the compound of Formula (I) is administered at a dose greater than 30 mg/m². In some embodiments, the compound of Formula (I) is administered at a dose of about 40 mg/m². In some embodiments, the compound of Formula (I) is administered on day 1 of a 14 day dosing cycle. In some embodiments, the compound of Formula (I) is administered on day 1 of a 21 day dosing cycle. In some embodiments, the compound of Formula (I) is selected from plinabulin, (3Z,6Z)-3-(phenyl-2,3,4,5,6-d₅)-methylene-6-((5-(tert-butyl)-1H-imidazol-4-yl)methylene)piperazine-2,5-dione; (3Z,6Z)-3-(phenyl-2,3,4,5,6-d₅)-methylene-d-6-((5-(tert-butyl)-1H-imidazol-4-yl)methylene)piperazine-2,5-dione; (3Z,6Z)-3-(phenylmethylene-d)-6-((5-(tert-butyl)-1H-imidazol-4-yl)methylene-d)piperazine-2,5-dione; (3Z,6Z)-3-(phenyl-2,3,4,5,6-d₅)-methylene-6-((5-(tert-butyl)-1H-imidazol-4-yl)methylene-d)piperazine-2,5-dione; (3Z,6Z)-3-(phenylmethylene)-6-((5-(tert-butyl)-1H-imidazol-4-yl)methylene-d)piperazine-2,5-dione; (3Z,6Z)-3-(phenyl-2,3,4,5,6-d₅)-methylene-d-6-((5-(tert-butyl)-1H-imidazol-4-yl)methylene-d)piperazine-2,5-dione; (3Z,6Z)-3-(4-Fluoro-(phenyl-2,3,5,6-d₄))-methylene-6-((5-(tert-butyl)-1H-imidazol-4-yl)methylene)piperazine-2,5-dione; (3Z,6Z)-3-(4-Fluoro-(phenyl-2,3,5,6-d₄))-methylene-6-((5-(tert-butyl)-1H-imidazol-4-yl)methylene-d)piperazine-2,5-dione; (3Z,6Z)-3-(3-fluorobenzylidene)-6-((5-(tert-butyl)-1H-imidazol-4-yl)methylene-d)piperazine-2,5-dione; (3Z,6Z)-3-(3-benzoylbenzylidene)-6-((5-(tert-butyl)-1H-imidazol-4-yl)methylene-d)piperazine-2,5-dione; (3Z,6Z)-3-(3-(4-fluorobenzoyl)benzylidene)-6-((5-(tert-butyl)-1H-imidazol-4-yl)methylene-d)piperazine-2,5-dione; (3Z,6Z)-3-(3-(4-methoxybenzoyl)benzylidene)-6-((5-(tert-butyl)-1H-imidazol-4-yl)methylene-d)piperazine-2,5-dione; (3Z,6Z)-3-(3-methoxybenzylidene)-6-((5-(tert-butyl)-1H-imidazol-4-yl)methylene-d)piperazine-2,5-dione; (3Z,6Z)-3-(3-(trifluoromethyenzydene)-6-((5-(tert-butyl)-1H-imidazol-4-yl)methylene-d)piperazine-2,5-dione; and a pharmaceutically acceptable salt thereof. In some embodiments, the compound of Formula (I) is plinabulin or a pharmaceutically acceptable salt thereof. In some embodiments, the cancer includes a tumor and a mass of the tumor is reduced from about 50% to about 100%. In some embodiments, the tumor mass is reduced from about 50% to about 70%.

Some aspects relate to a method of halting or reversing progressive cancer in a subject. In some embodiments, the method includes administering a compound of Formula (I) as a monotherapy,

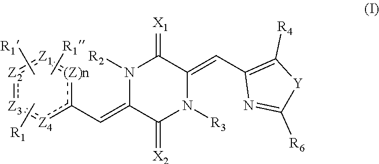

or a pharmaceutically acceptable salt thereof, wherein:
R₁, R₄, and R₆, are each separately selected from the group consisting of a hydrogen atom, a deuterium atom, a halogen atom, and saturated C₁-C₂₄ alkyl, unsaturated C₁-C₂₄ alkenyl, cycloalkyl, cycloalkenyl, alkoxy, cycloalkoxy, aryl, substituted aryl, heteroaryl, substituted heteroaryl, amino, substituted amino, nitro, azido, substituted nitro, phenyl, and substituted phenyl groups, hydroxy, carboxy, —CO—O—R₇, cyano, alkylthio, halogenated alkyl including polyhalogenated alkyl, halogenated carbonyl, and carbonyl —CH₂CO—R₇, wherein R₇ is selected from a hydrogen atom, a halogen atom, and saturated C₁-C₂₄ alkyl, unsaturated C₁-C₂₄ alkenyl, cycloalkyl, cycloalkenyl, alkoxy, cycloalkoxy, aryl, substituted aryl, heteroaryl, substituted heteroaryl, amino, substituted amino, nitro, azido, substituted nitro, phenyl, and substituted phenyl groups;
R₁′ and R₁″ are each independently selected from the group consisting of a hydrogen atom, a deuterium atom, a halogen atom, and saturated C₁-C₂₄ alkyl, unsaturated C₁-C₂₄ alkenyl, cycloalkyl, cycloalkenyl, alkoxy, cycloalkoxy, aryl, substituted aryl, heteroaryl, substituted heteroaryl, amino, substituted amino, nitro, azido, substituted nitro, phenyl, and substituted phenyl groups, hydroxy, carboxy, —CO—O—R₇, cyano, alkylthio, halogenated alkyl including polyhalogenated alkyl, halogenated carbonyl, and carbonyl —CH₂CO—R₇, wherein R₇ is selected from a hydrogen atom, a halogen atom, and saturated C₁-C₂₄ alkyl, unsaturated C₁-C₂₄ alkenyl, cycloalkyl, cycloalkenyl, alkoxy, cycloalkoxy, aryl, substituted aryl, heteroaryl, substituted heteroaryl, amino, substituted amino, nitro, azido, substituted nitro, phenyl, and substituted phenyl groups;
R, R₁′ and R₁″ are either covalently bound to one another or are not covalently bound to one another;
R₂, R₃, and R₅ are each separately selected from the group consisting of a hydrogen atom, a deuterium atom, a halogen atom, and saturated C₁-C₁₂ alkyl, unsaturated C₁-C₁₂alkenyl, acyl, cycloalkyl, alkoxy, cycloalkoxy, aryl, substituted aryl, heteroaryl, substituted heteroaryl, amino, substituted amino, nitro, and substituted nitro groups, sulfonyl and substituted sulfonyl groups;
m is an integer equal to zero, one or two;
X₁ and X₂ are separately selected from the group consisting of an oxygen atom, a nitrogen atom and a sulfur atom, and
Y is selected from the group consisting NRs, an oxygen atom, a sulfur atom, a oxidized sulfur atom, a methylene group and a substituted methylene group;
Z, for each separate n, if non-zero, and Z₁, Z₂, Z₃ and Z₄ are each separately selected from a carbon atom, a sulfur atom, a nitrogen atom or an oxygen atom;
the dashed bonds may be either single or double bonds,
wherein the cancer is selected from gastric cancer, small cell lung cancer, osteosarcoma, bladder cancer, and triple negative breast cancer.

In some embodiments, the compound of Formula (I) is plinabulin or a pharmaceutically acceptable salt thereof.

Some aspects relate to a method of inhibiting proliferation of a cancer cell. In some embodiments, the method includes contacting the cancer cell with an effective amount of plinabulin and no other chemotherapeutic agent, wherein the cancer cell is from a cancer selected from gastric cancer, small cell lung cancer, osteosarcoma, bladder cancer, and triple negative breast cancer.

Some aspects relate to a method of inducing apoptosis in a cancer cell. In some embodiments, the method includes contacting the cancer cell with an effective amount of plinabulin and no other chemotherapeutic agent, wherein the cancer is selected from gastric cancer, small cell lung cancer, osteosarcoma, bladder cancer, and triple negative breast cancer.

### BRIEF DESCRIPTION OF THE DRAWINGS

FIG. 1 is a graph illustrating the efficacy of plinabulin monohydrate in a clonogenic assay.

FIG. 2 is a heatmap illustrating the IC₇₀ values of a study ranked to absolute IC₇₀.

FIG. 3 is a line graph illustrating the concentration-effect curves for plinabulin monohydrate and small cell lung cancer.

FIG. 4 is a line graph illustrating the concentration-effect curves for plinabulin monohydrate and gastric cancer.

FIG. 5 is a line graph illustrating the concentration-effect curves for plinabulin monohydrate and triple negative breast cancer.

FIG. 6 is a graph illustrating the efficacy of plinabulin monohydrate in a clonogenic assay with IC₅₀ values.

FIG. 7 is a graph illustrating the efficacy of plinabulin monohydrate in a clonogenic assay with IC₇₀ values.

FIG. 8 is a heatmap illustrating the IC₅₀ values of a study ranked to absolute IC₅₀.

FIG. 9 is a heatmap illustrating the IC₇₀ values of a study ranked to absolute IC₇₀.

FIG. 10 is a line graph illustrating the concentration-effect curves for plinabulin monohydrate and small cell lung cancer.

FIG. 11 is a line graph illustrating the concentration-effect curves for plinabulin monohydrate and gastric cancer.

FIG. 12 is a line graph illustrating the concentration-effect curves for plinabulin monohydrate and triple negative breast cancer.

FIG. 13A is a heatmap illustrating the IC₅₀/IC₇₀ values obtained for plinabulin; FIG. 13B is a continuation of the heatmap from FIG. 13A.

FIG. 14A is a heatmap illustrating T/C values obtained for plinabulin; FIG. 14B is a continuation of the heatmap from FIG. 14A.

### DETAILED DESCRIPTION

The present disclosure provides methods for treating a cancer or a tumor. Some embodiments relate to using Plinabulin to treat a cancer or a tumor, including but not limited to small cell lung cancer, gastric cancer, and triple negative breast cancer. In some embodiments, methods provided herein are useful in treating, delaying the progression of, preventing relapse of, or alleviating a symptom of a cancer or a tumor, including but not limited to small cell lung cancer, gastric cancer, and triple negative breast cancer. In some embodiments, the compound of Formula (I) is plinabulin. Plinabulin, (3Z,6Z)-3-Benzylidene-6-{[5-(2-methyl-2-propanyl)-1H-imidazol-4-yl]methylene}-2,5-piperazinedione, is a synthetic analog of the natural compound phenylahistin. As described herein, it was surprisingly discovered that plinabulin can be effective as a monotherapy against a cancer or a tumor, including but not limited to small cell lung cancer, gastric cancer, and triple negative breast cancer.

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Methods recited herein may be carried out in any order of the recited events, which is logically possible, as well as the recited order of events.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

The term “agent” is used herein to denote a chemical compound, a mixture of chemical compounds, a biological macromolecule, or an extract made from biological materials.

The term “ameliorate” as used herein refers to any reduction in the extent, severity, frequency, and/or likelihood of a symptom or clinical sign characteristic of a particular condition.

The terms “cancer”, “neoplasm”, and “carcinoma”, are used interchangeably herein to refer to cells which exhibit relatively autonomous growth, so that they exhibit an aberrant growth phenotype characterized by a significant loss of control of cell proliferation. In general, cells of interest for detection or treatment in the present application include precancerous (e.g., benign), malignant, pre-metastatic, metastatic, and non-metastatic cells. Detection of cancerous cells is of particular interest.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety. The pharmaceutically acceptable excipient can be a monosaccharide or monosaccharide derivative.

The term “subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate.

The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea pigs, or the like.

The terms “effective amount” or a “therapeutically effective amount” as used herein refers to an amount of a therapeutic agent that is effective to relieve, to some extent, or to reduce the likelihood of onset of, one or more of the symptoms of a disease or condition, and can include curing a disease or condition.

The terms “treat,” “treatment,” or “treating,” as used herein refers to administering a compound or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

As used herein, the term “chemotherapeutic agent” refers to an agent that reduces, prevents, mitigates, limits, and/or delays the growth of metastases or neoplasms, or kills neoplastic cells directly by necrosis or apoptosis of neoplasms or any other mechanism, or that can be otherwise used, in a pharmaceutically-effective amount, to reduce, prevent, mitigate, limit, and/or delay the growth of metastases or neoplasms in a subject with neoplastic disease. Chemotherapeutic agents include but are not limited to, for example, fluoropyrimidines; pyrimidine nucleosides; purine nucleosides; anti-folates, platinum-based agents; anthracyclines/anthracenediones; epipodophyllotoxins; camptothecins; hormones; hormonal complexes; antihormonals; enzymes, proteins, peptides and polyclonal and/or monoclonal antibodies; vinca alkaloids; taxanes; epothilones; antimicrotubule agents; alkylating agents; antimetabolites; topoisomerase inhibitors; antivirals; and various other cytotoxic and cytostatic agents.

### Compounds

In some embodiments, the compounds and therapeutic compositions for treating a cancer or tumor described herein include a compound represented by Formula (I):

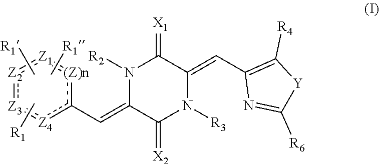

wherein
R₁, R₄, and R₆, are each separately selected from the group consisting of a hydrogen atom, a deuterium atom, a halogen atom, and saturated C₁,—C₂₄ alkyl, unsaturated C₁-C₂₄ alkenyl, cycloalkyl, cycloalkenyl, alkoxy, cycloalkoxy, aryl, substituted aryl, heteroaryl, substituted heteroaryl, amino, substituted amino, nitro, azido, substituted nitro, phenyl, and substituted phenyl groups, hydroxy, carboxy, —CO—O—R₇, cyano, alkylthio, halogenated alkyl including polyhalogenated alkyl, halogenated carbonyl, and carbonyl —CH₂CO—R₇, wherein R₇ is selected from a hydrogen atom, a halogen atom, and saturated C₁-C₂₄ alkyl, unsaturated C₁-C₂₄ alkenyl, cycloalkyl, cycloalkenyl, alkoxy, cycloalkoxy, aryl, substituted aryl, heteroaryl, substituted heteroaryl, amino, substituted amino, nitro, azido, substituted nitro, phenyl, and substituted phenyl groups;
R₁′ and R₁″ are each independently selected from the group consisting of a hydrogen atom, a deuterium atom, a halogen atom, and saturated C₁-C₂₄ alkyl, unsaturated C₁-C₂₄ alkenyl, cycloalkyl, cycloalkenyl, alkoxy, cycloalkoxy, aryl, substituted aryl, heteroaryl, substituted heteroaryl, amino, substituted amino, nitro, azido, substituted nitro, phenyl, and substituted phenyl groups, hydroxy, carboxy, —CO—O—R₇, cyano, alkylthio, halogenated alkyl including polyhalogenated alkyl, halogenated carbonyl, and carbonyl —CH₂CO—R₇, wherein R₇ is selected from a hydrogen atom, a halogen atom, and saturated C₁-C₂₄ alkyl, unsaturated C₁-C₂₄ alkenyl, cycloalkyl, cycloalkenyl, alkoxy, cycloalkoxy, aryl, substituted aryl, heteroaryl, substituted heteroaryl, amino, substituted amino, nitro, azido, substituted nitro, phenyl, and substituted phenyl groups;
R, R₁′ and R₁″ are either covalently bound to one another or are not covalently bound to one another;
R₂, R₃, and R₅ are each separately selected from the group consisting of a hydrogen atom, a deuterium atom, a halogen atom, and saturated C₁-C₁₂ alkyl, unsaturated C₁-C₁₂ alkenyl, acyl, cycloalkyl, alkoxy, cycloalkoxy, aryl, substituted aryl, heteroaryl, substituted heteroaryl, amino, substituted amino, nitro, and substituted nitro groups, sulfonyl and substituted sulfonyl groups;
m is an integer equal to zero, one or two;
X₁ and X₂ are separately selected from the group consisting of an oxygen atom, a nitrogen atom and a sulfur atom, and
Y is selected from the group consisting of NRs, an oxygen atom, a sulfur atom, a oxidized sulfur atom, a methylene group and a substituted methylene group;
Z, for each separate n, if non-zero, and Z₁, Z₂, Z₃ and Z₄ are each separately selected from a carbon atom, a sulfur atom, a nitrogen atom or an oxygen atom; and the dashed bonds may be either single or double bonds.

A compound of Formula (I) can be readily prepared according to methods and procedures detailed in U.S. Pat. Nos. 7,064,201 and 7,919,497, which are incorporated herein by reference in their entireties.

In some embodiments, the compounds described herein are a dehydrophenylahistin represented by Formula (II):

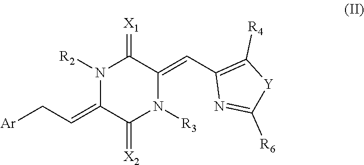

wherein
R₂ and R₃ are each separately selected from the group consisting of a hydrogen atom; a halogen atom; mono-substituted; poly-substituted or unsubstituted, straight or branched chain variants of the following residues: C₁-C₁₂ alkyl, C₁-C₁₂ alkenyl, acyl, and alkoxy; and mono-substituted, poly-substituted or unsubstituted variants of the following residues: cycloalkyl, cycloalkoxy, aryl, heteroaryl, amino, nitro, and sulfonyl; or R₂ is a bond to Ar;
R₄ and R₆ are each separately selected from the group consisting of hydrogen; halogen; hydroxyl; mono-substituted, poly-substituted or unsubstituted, straight or branched chain variants of the following residues: C₁-C₂₄ alkyl, C₂-C₂₄ alkenyl, C₂-C₂₄ alkynyl, alkoxy, acyl, arylalkyl, heteroarylalkyl, alkyloxycarbonyloxy, ester, arylalkoxy, alkoxy, and alkylthio; mono-substituted, poly-substituted or unsubstituted variants of the following residues: acyloxy, aryloxycarbonyloxy, cycloalkyl, cycloalkenyl, cycloalkoxy, aryl, heteroaryl, aryloxy, arylcarbonyl, heterocycloalkyl, carbonyl, amino, aminocarbonyl, amide, aminocarbonyloxy, nitro, azido, phenyl, hydroxyl, thio, alkylthio, arylthio, thiooxysulfonyl, thiophene, carboxy, and cyano;
X₁ and X₂ are separately selected from the group consisting of an oxygen atom, a sulfur atom, and a nitrogen atom substituted with a R₅ group;
R₅ is selected from the group consisting of a hydrogen atom, a halogen atom, and saturated C₁-C₁₂ alkyl, unsaturated C₁-C₁₂ alkenyl, acyl, cycloalkyl, alkoxy, cycloalkoxy, aryl, substituted aryl, heteroaryl, substituted heteroaryl, amino, substituted amino, nitro, and substituted nitro groups, sulfonyl and substituted sulfonyl groups;
Y is selected from the group consisting of NRs, an oxygen atom, a sulfur atom, an oxidized sulfur atom, a methylene group, and a substituted methylene group;
n is 0, 1, 2, 3, or 4; and
Ar is a cyclic or polycyclic aryl or heteroaryl ring system comprising between one and three rings, wherein:
each ring in said system is separately a 5, 6, 7, or 8 membered ring;
each ring in said system separately comprises 0, 1, 2, 3, or 4 heteroatoms selected from the group consisting of oxygen, sulfur, and nitrogen; and
each ring in said system is optionally substituted with one or more substituents selected from the group consisting of hydrogen; halogen; hydroxyl; mono-substituted, poly-substituted or unsubstituted, straight or branched chain variants of the following residues: C₁-C₂₄ alkyl, C₂-C₂₄ alkenyl, C₂-C₂₄ alkynyl, alkoxy, acyl, arylalkyl, heteroarylalkyl, alkyloxycarbonyloxy, ester, arylalkoxy, alkoxy, and alkylthio; mono-substituted, poly-substituted or unsubstituted variants of the following residues: acyloxy, aryloxycarbonyloxy, cycloalkyl, cycloalkenyl, cycloalkoxy, aryl, heteroaryl, aryloxy, arylcarbonyl, heterocycloalkyl, carbonyl, amino, aminocarbonyl, amide, aminocarbonyloxy, nitro, azido, phenyl, hydroxyl, thio, alkylthio, arylthio, thiophene, oxysulfonyl, sulfonyl, carboxy, and cyano; and an optionally substituted fused ring selected from the group consisting of dioxole, dithiole, oxathiole, dioxine, dithiine, and oxathiine.

A compound of Formula (II) can be readily prepared according to methods and procedures detailed in U.S. Pat. Nos. 7,064,201 and 7,919,497, which are incorporated herein by reference in their entireties.

In some embodiments, a compound of Formula (I) is selected from plinabulin, (3Z,6Z)-3-(phenyl-2,3,4,5,6-d₅)-methylene-6-((5-(tert-butyl)-1H-imidazol-4-yl)methylene)piperazine-2,5-dione; (3Z,6Z)-3-(phenyl-2,3,4,5,6-d₅)-methylene-d-6-((5-(tert-butyl)-1H-imidazol-4-yl)methylene)piperazine-2,5-dione; (3Z,6Z)-3-(phenylmethylene-d)-6-((5-(tert-butyl)-1H-imidazol-4-yl)methylene-d)piperazine-2,5-dione; (3Z,6Z)-3-(phenyl-2,3,4,5,6-d₅)-methylene-6-((5-(tert-butyl)-1H-imidazol-4-yl)methylene-d)piperazine-2,5-dione; (3Z,6Z)-3-(phenylmethylene)-6-((5-(tert-butyl)-1H-imidazol-4-yl)methylene-d)piperazine-2,5-dione; (3Z,6Z)-3-(phenyl-2,3,4,5,6-d₅)-methylene-d-6-((5-(tert-butyl)-1H-imidazol-4-yl)methylene-d)piperazine-2,5-dione; (3Z,6Z)-3-(4-Fluoro-(phenyl-2,3,5,6-d₄))-methylene-6-((5-(tert-butyl)-1H-imidazol-4-yl)methylene)piperazine-2,5-dione; (3Z,6Z)-3-(4-Fluoro-(phenyl-2,3,5,6-d₄))-methylene-6-((5-(tert-butyl)-1H-imidazol-4-yl)methylene-d)piperazine-2,5-dione; (3Z,6Z)-3-(3-fluorobenzylidene)-6-((5-(tert-butyl)-1H-imidazol-4-yl)methylene-d)piperazine-2,5-dione; (3Z,6Z)-3-(3-benzoylbenzylidene)-6-((5-(tert-butyl)-1H-imidazol-4-yl)methylene-d)piperazine-2,5-dione; (3Z,6Z)-3-(3-(4-fluorobenzoyl)benzylidene)-6-((5-(tert-butyl)-1H-imidazol-4-yl)methylene-d)piperazine-2,5-dione; (3Z,6Z)-3-(3-(4-methoxybenzoyl)benzylidene)-6-((5-(tert-butyl)-1H-imidazol-4-yl)methylene-d)piperazine-2,5-dione; (3Z,6Z)-3-(3-methoxybenzylidene)-6-((5-(tert-butyl)-1H-imidazol-4-yl)methylene-d)piperazine-2,5-dione; (3Z,6Z)-3-(3-(trifluoromethyenzydene)-6-((5-(tert-butyl)-1H-imidazol-4-yl)methylene-d)piperazine-2,5-dione; and pharmaceutically acceptable salts thereof.

In some embodiments, the compound of Formula (I) is plinabulin. In some embodiments, the compound of Formula (I) is plinabulin monohydrate. In some embodiments, the compound of Formula (I) is a salt form of plinabulin. Plinabulin can be readily prepared according to methods and procedures detailed in U.S. Pat. Nos. 7,064,201 and 7,919,497, which are incorporated herein by reference in their entireties.

### Use and Method of Treatment

In aspects, the present disclosure provides methods and therapeutic compositions for treating, preventing, or ameliorating a cancer or tumor in a subject by administering a compound of Formula (I) (e.g., plinabulin), or a pharmaceutically acceptable salt thereof, as a monotherapy. In some embodiments, the cancer or tumor is selected from gastric cancer, small cell lung cancer, osteosarcoma, bladder cancer, and triple negative breast cancer. In some embodiments, the cancer or tumor is gastric cancer. In some embodiments, the cancer or tumor is small cell lung cancer. In some embodiments, the cancer or tumor is triple negative breast cancer.

In some embodiments, the method comprises administering the compound of Formula (I) (e.g., plinabulin) at a dose from about 5 mg/m² to 150 mg/m². In some embodiments, the method comprises administering the compound of Formula (I) (e.g., plinabulin) at a dose from about 10 mg/m² to 50 mg/m². In some embodiments, the method comprises administering the compound of Formula (I) (e.g., plinabulin) at a dose from about 20 mg/m² to 30 mg/m². In some embodiments, the compound of Formula (I) (e.g., plinabulin) is administered at a dose that is greater than 20 mg/m². In some embodiments, the compound of Formula (I) (e.g., plinabulin) is administered at a dose that is greater than 30 mg/m². In some embodiments, the compound of Formula (I) (e.g., plinabulin) is administered at a dose of about 40 mg/m². In some embodiments, plinabulin is administered at a dose of from about 30 mg to about 40 mg. In some embodiments, plinabulin is administered at a dose of about 40 mg.

In some embodiments, the compound of Formula (I) (e.g., plinabulin) is administered on day 1 of a 14 day dosing cycle. In some embodiments, the compound of Formula (I) (e.g., plinabulin) is administered on day 1 of a 21 day dosing cycle.

Some embodiments relate to a method of halting or reversing a progressive cancer in a subject. In some embodiments, the method comprises administering a compound of Formula (I) to the subject. In some embodiments, the cancer cell is from a cancer selected from gastric cancer, small cell lung cancer, osteosarcoma, bladder cancer, and triple negative breast cancer. In some embodiments, the compound of Formula (I) is plinabulin or a pharmaceutically acceptable salt thereof.

Some embodiments relate to a method of inhibiting the proliferation of a cancer cell. In some embodiments, the method comprises contacting the cancer cell with a compound of Formula (I) to the subject. In some embodiments, the cancer cell is from a cancer selected from gastric cancer, small cell lung cancer, osteosarcoma, bladder cancer, and triple negative breast cancer. In some embodiments, the compound of Formula (I) is plinabulin or a pharmaceutically acceptable salt thereof.

Some embodiments relate to a method of inducing apoptosis in a cancer cell. In some embodiments, the method comprises contacting the cancer cell with a compound of Formula (I) to the subject. In some embodiments, the cancer cell is from a cancer selected from gastric cancer, small cell lung cancer, osteosarcoma, bladder cancer, and triple negative breast cancer. In some embodiments, the compound of Formula (I) is plinabulin or a pharmaceutically acceptable salt thereof.

Some embodiments relate to a method of inhibiting the progression of a cancer. In some embodiments, the method comprises administering an effective amount of Plinabulin to a subject in need thereof.

### Administration

Administration of the pharmaceutical compositions described herein can be via any of the accepted modes of administration for agents that serve similar utilities including, but not limited to, orally, sublingually, buccally, subcutaneously, intravenously, intranasally, intratumorally, topically, transdermally, intradermally, intraperitoneally, intramuscularly, intrapulmonarily, vaginally, rectally, or intraocularly. Oral and parenteral administrations are customary in treating the indications that are the subject of the preferred embodiments.

The compositions described herein may be provided in unit dosage form. As used herein, a “unit dosage form” is a composition containing an amount of a compound or composition that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as an infusion over a period of time (e.g., from about 30 minutes to about 2-6 hours), or administered as a continuous infusion, and may be given more than once during a course of therapy, although a single administration is not specifically excluded. The skilled artisan will recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, sublingual, buccal, nasal, rectal, topical (including transdermal and intradermal), ocular, intracerebral, intracranial, intrathecal, intra-arterial, intravenous, intramuscular, or other parental routes of administration. The skilled artisan will appreciate that oral and nasal compositions include compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the inhibitory activity of the compound or composition. The amount of carrier employed in conjunction with the compound or composition is sufficient to provide a practical quantity of material for administration per unit dose of the compound. Techniques and compositions for making dosage forms useful in the methods described herein are described in the following references, all incorporated by reference herein: Modem Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules (e.g. solid gel capsules and liquid gel capsules), granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carriers suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmellose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject composition is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include additional drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort may be maximized as much as possible, although sometimes formulation considerations (e.g. drug stability) may necessitate less than optimal comfort. In the case that comfort cannot be maximized, the liquid may be formulated such that the liquid is tolerable to the patient for topical ophthalmic use. Additionally, an ophthalmically acceptable liquid may either be packaged for single use, or contain a preservative to prevent contamination over multiple uses.

For ophthalmic application, solutions or medicaments are often prepared using a physiological saline solution as a major vehicle. Ophthalmic solutions may preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

Ophthalmically acceptable antioxidants include, but are not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, creams, ointments, gels, solutions or suspensions, etc., containing the composition disclosed herein are employed. Topical formulations may generally be comprised of a pharmaceutical carrier, co-solvent, emulsifier, penetration enhancer, preservative system, and emollient.

For intravenous administration, the compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. In some embodiments, excipients utilized for intravenous delivery may include Kolliphor HS 15 (polyoxyl 15 hydroxystearate or Solutol HS-15), propylene glycol and 5% dextrose in water (D5W). Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol. In some embodiments, plinabulin is added to a light protected bag containing at least 200 ml D5W to achieve 0.08 mg/ml-0.2 mg/ml plinabulin. In some embodiments, 40 mg of plinabulin crude drug was taken and was added to 6.0 g of propylene glycol, which was then added to 200 ml of D5W. In some embodiments, plinabulin is a concentrated solution (4 mg/ml plinabulin in propylene glycol/polyoxyl 15 hydroxystearates, 60:40 (wt:wt)). In some embodiments, the plinabulin in a concentrated solution is added to a D5W injection (e.g., a 1:10 dilution level or a 1:20 dilution level).

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The actual dose of the active compounds described herein depends on the specific compound, and on the condition to be treated; the selection of the appropriate dose is well within the knowledge of the skilled artisan. In some embodiments, a compound of Formula (I) may be administered at a dose in the range of about 1 mg/m² to about 50 mg/m². In some embodiments, a compound of Formula (I) is administered at a dose in the range of about 1-50 mg/m² of the body surface area. In some embodiments, a compound of Formula (I) is administered at a dose in the range of about 1-2, 1-3, 1-4, 1-5, 1-6, 1-7, 1-8, 1-9, 1-10, 1-11, 1-12, 1-13, 1-13.75, 1-14, 1-15, 1-16, 1-17, 1-18, 1-19, 1-20, 1-22.5, 1-25, 1-27.5, 1-30, 1.5-2, 1.5-3, 1.5-4, 1.5-5, 1.5-6, 1.5-7, 1.5-8, 1.5-9, 1.5-10, 1.5-11, 1.5-12, 1.5-13, 1.5-13.75, 1.5-14, 1.5-15, 1.5-16, 1.5-17, 1.5-18, 1.5-19, 1.5-20, 1.5-22.5, 1.5-25, 1.5-27.5, 1.5-30, 2.5-2, 2.5-3, 2.5-4, 2.5-5, 2.5-6, 2.5-7, 2.5-8, 2.5-9, 2.5-10, 2.5-11, 2.5-12, 2.5-13, 2.5-13.75, 2.5-14, 2.5-15, 2.5-16, 2.5-17, 2.5-18, 2.5-19, 2.5-20, 2.5-22.5, 2.5-25, 2.5-27.5, 2.5-30, 2.5-7.5, 3-4, 3-5, 3-6, 3-7, 3-8, 3-9, 3-10, 3-11, 3-12, 3-13, 3-13.75, 3-14, 3-15, 3-16, 3-17, 3-18, 3-19, 3-20, 3-22.5, 3-25, 3-27.5, 3-30, 3.5-6.5, 3.5-13.75, 3.5-15, 2.5-17.5, 4-5, 4-6, 4-7, 4-8, 4-9, 4-10, 4-11, 4-12, 4-13, 4-13.75, 4-14, 4-15, 4-16, 4-17, 4-18, 4-19, 4-20, 4-22.5, 4-25, 4-27.5, 4-30, 5-6, 5-7, 5-8, 5-9, 5-10, 5-11, 5-12, 5-13, 5-13.75, 5-14, 5-15, 5-16, 5-17, 5-18, 5-19, 5-20, 5-22.5, 5-25, 5-27.5, 5-30, 6-7, 6-8, 6-9, 6-10, 6-11, 6-12, 6-13, 6-13.75, 6-14, 6-15, 6-16, 6-17, 6-18, 6-19, 6-20, 6-22.5, 6-25, 6-27.5, 6-30, 7-8, 7-9, 7-10, 7-11, 7-12, 7-13, 7-13.75, 7-14, 7-15, 7-16, 7-17, 7-18, 7-19, 7-20, 7-22.5, 7-25, 7-27.5, 7-30, 7.5-12.5, 7.5-13.5, 7.5-15, 8-9, 8-10, 8-11, 8-12, 8-13, 8-13.75, 8-14, 8-15, 8-16, 8-17, 8-18, 8-19, 8-20, 8-22.5, 8-25, 8-27.5, 8-30, 9-10, 9-11, 9-12, 9-13, 9-13.75, 9-14, 9-15, 9-16, 9-17, 9-18, 9-19, 9-20, 9-22.5, 9-25, 9-27.5, 9-30, 10-11, 10-12, 10-13, 10-13.75, 10-14, 10-15, 10-16, 10-17, 10-18, 10-19, 10-20, 10-22.5, 10-25, 10-27.5, 10-30, 11.5-15.5, 12.5-14.5, 7.5-22.5, 8.5-32.5, 9.5-15.5, 15.5-24.5, 5-35, 17.5-22.5, 22.5-32.5, 25-35, 25.5-34.5, 27.5-32.5, 2-20, 2.5-22.5, 9.5-21.5, 10-50, 15-50, 20-50, 25-50, 30-50, 35-50, or 40-50 mg/m², of the body surface area. In some embodiments, a compound of Formula (I) is administered at a dose of about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40, 45, 50 mg/m² of the body surface area. In some embodiments, a compound of Formula (I) is administered at a dose less than about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40, 45, 50 mg/m² of the body surface area. In some embodiments, a compound of Formula (I) is administered at a dose greater than about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40, 41, 42, 43, 44, 45, 46, 47, 48, 49, 50 mg/m² of the body surface area. In some embodiments, a compound of Formula (I) is administered at a dose of about 40 mg/m² of the body surface area.

In some embodiments, a compound of Formula (I) dose is about 5 mg-300 mg, 5 mg-200 mg, 7.5 mg-200 mg, 10 mg-100 mg, 15 mg-100 mg, 20 mg-100 mg, 30 mg-100 mg, 40 mg-100 mg, 10 mg-80 mg, 15 mg-80 mg, 20 mg-80 mg, 30 mg-80 mg, 40 mg-80 mg, 10 mg-60 mg, 15 mg-60 mg, 20 mg-60 mg, 30 mg-60 mg, or about 40 mg-60 mg. In some embodiments, a compound of Formula (I) administered is about 20 mg-60 mg, 27 mg-60 mg, 20 mg-45 mg, or 27 mg-45 mg. In some embodiments, a compound of Formula (I) administered is about 5 mg-7.5 mg, 5 mg-9 mg, 5 mg-10 mg, 5 mg-12 mg, 5 mg-14 mg, 5 mg-15 mg, 5 mg-16 mg, 5 mg-18 mg, 5 mg-20 mg, 5 mg-22 mg, 5 mg-24 mg, 5 mg-26 mg, 5 mg-28 mg, 5 mg-30 mg, 5 mg-32 mg, 5 mg-34 mg, 5 mg-36 mg, 5 mg-38 mg, 5 mg-40 mg, 5 mg-42 mg, 5 mg-44 mg, 5 mg-46 mg, 5 mg-48 mg, 5 mg-50 mg, 5 mg-52 mg, 5 mg-54 mg, 5 mg-56 mg, 5 mg-58 mg, 5 mg-60 mg, 7 mg-7.7 mg, 7 mg-9 mg, 7 mg-10 mg, 7 mg-12 mg, 7 mg-14 mg, 7 mg-15 mg, 7 mg-16 mg, 7 mg-18 mg, 7 mg-20 mg, 7 mg-22 mg, 7 mg-24 mg, 7 mg-26 mg, 7 mg-28 mg, 7 mg-30 mg, 7 mg-32 mg, 7 mg-34 mg, 7 mg-36 mg, 7 mg-38 mg, 7 mg-40 mg, 7 mg-42 mg, 7 mg-44 mg, 7 mg-46 mg, 7 mg-48 mg, 7 mg-50 mg, 7 mg-52 mg, 7 mg-54 mg, 7 mg-56 mg, 7 mg-58 mg, 7 mg-60 mg, 9 mg-10 mg, 9 mg-12 mg, 9 mg-14 mg, 9 mg-15 mg, 9 mg-16 mg, 9 mg-18 mg, 9 mg-20 mg, 9 mg-22 mg, 9 mg-24 mg, 9 mg-26 mg, 9 mg-28 mg, 9 mg-30 mg, 9 mg-32 mg, 9 mg-34 mg, 9 mg-36 mg, 9 mg-38 mg, 9 mg-40 mg, 9 mg-42 mg, 9 mg-44 mg, 9 mg-46 mg, 9 mg-48 mg, 9 mg-50 mg, 9 mg-52 mg, 9 mg-54 mg, 9 mg-56 mg, 9 mg-58 mg, 9 mg-60 mg, 10 mg-12 mg, 10 mg-14 mg, 10 mg-15 mg, 10 mg-16 mg, 10 mg-18 mg, 10 mg-20 mg, 10 mg-22 mg, 10 mg-24 mg, 10 mg-26 mg, 10 mg-28 mg, 10 mg-30 mg, 10 mg-32 mg, 10 mg-34 mg, 10 mg-36 mg, 10 mg-38 mg, 10 mg-40 mg, 10 mg-42 mg, 10 mg-44 mg, 10 mg-46 mg, 10 mg-48 mg, 10 mg-50 mg, 10 mg-52 mg, 10 mg-54 mg, 10 mg-56 mg, 10 mg-58 mg, 10 mg-60 mg, 12 mg-14 mg, 12 mg-15 mg, 12 mg-16 mg, 12 mg-18 mg, 12 mg-20 mg, 12 mg-22 mg, 12 mg-24 mg, 12 mg-26 mg, 12 mg-28 mg, 12 mg-30 mg, 12 mg-32 mg, 12 mg-34 mg, 12 mg-36 mg, 12 mg-38 mg, 12 mg-40 mg, 12 mg-42 mg, 12 mg-44 mg, 12 mg-46 mg, 12 mg-48 mg, 12 mg-50 mg, 12 mg-52 mg, 12 mg-54 mg, 12 mg-56 mg, 12 mg-58 mg, 12 mg-60 mg, 15 mg-16 mg, 15 mg-18 mg, 15 mg-20 mg, 15 mg-22 mg, 15 mg-24 mg, 15 mg-26 mg, 15 mg-28 mg, 15 mg-30 mg, 15 mg-32 mg, 15 mg-34 mg, 15 mg-36 mg, 15 mg-38 mg, 15 mg-40 mg, 15 mg-42 mg, 15 mg-44 mg, 15 mg-46 mg, 15 mg-48 mg, 15 mg-50 mg, 15 mg-52 mg, 15 mg-54 mg, 15 mg-56 mg, 15 mg-58 mg, 15 mg-60 mg, 17 mg-18 mg, 17 mg-20 mg, 17 mg-22 mg, 17 mg-24 mg, 17 mg-26 mg, 17 mg-28 mg, 17 mg-30 mg, 17 mg-32 mg, 17 mg-34 mg, 17 mg-36 mg, 17 mg-38 mg, 17 mg-40 mg, 17 mg-42 mg, 17 mg-44 mg, 17 mg-46 mg, 17 mg-48 mg, 17 mg-50 mg, 17 mg-52 mg, 17 mg-54 mg, 17 mg-56 mg, 17 mg-58 mg, 17 mg-60 mg, 20 mg-22 mg, 20 mg-24 mg, 20 mg-26 mg, 20 mg-28 mg, 20 mg-30 mg, 20 mg-32 mg, 20 mg-34 mg, 20 mg-36 mg, 20 mg-38 mg, 20 mg-40 mg, 20 mg-42 mg, 20 mg-44 mg, 20 mg-46 mg, 20 mg-48 mg, 20 mg-50 mg, 20 mg-52 mg, 20 mg-54 mg, 20 mg-56 mg, 20 mg-58 mg, 20 mg-60 mg, 22 mg-24 mg, 22 mg-26 mg, 22 mg-28 mg, 22 mg-30 mg, 22 mg-32 mg, 22 mg-34 mg, 22 mg-36 mg, 22 mg-38 mg, 22 mg-40 mg, 22 mg-42 mg, 22 mg-44 mg, 22 mg-46 mg, 22 mg-48 mg, 22 mg-50 mg, 22 mg-52 mg, 22 mg-54 mg, 22 mg-56 mg, 22 mg-58 mg, 22 mg-60 mg, 25 mg-26 mg, 25 mg-28 mg, 25 mg-30 mg, 25 mg-32 mg, 25 mg-34 mg, 25 mg-36 mg, 25 mg-38 mg, 25 mg-40 mg, 25 mg-42 mg, 25 mg-44 mg, 25 mg-46 mg, 25 mg-48 mg, 25 mg-50 mg, 25 mg-52 mg, 25 mg-54 mg, 25 mg-56 mg, 25 mg-58 mg, 25 mg-60 mg, 27 mg-28 mg, 27 mg-30 mg, 27 mg-32 mg, 27 mg-34 mg, 27 mg-36 mg, 27 mg-38 mg, 27 mg-40 mg, 27 mg-42 mg, 27 mg-44 mg, 27 mg-46 mg, 27 mg-48 mg, 27 mg-50 mg, 27 mg-52 mg, 27 mg-54 mg, 27 mg-56 mg, 27 mg-58 mg, 27 mg-60 mg, 30 mg-32 mg, 30 mg-34 mg, 30 mg-36 mg, 30 mg-38 mg, 30 mg-40 mg, 30 mg-42 mg, 30 mg-44 mg, 30 mg-46 mg, 30 mg-48 mg, 30 mg-50 mg, 30 mg-52 mg, 30 mg-54 mg, 30 mg-56 mg, 30 mg-58 mg, 30 mg-60 mg, 33 mg-34 mg, 33 mg-36 mg, 33 mg-38 mg, 33 mg-40 mg, 33 mg-42 mg, 33 mg-44 mg, 33 mg-46 mg, 33 mg-48 mg, 33 mg-50 mg, 33 mg-52 mg, 33 mg-54 mg, 33 mg-56 mg, 33 mg-58 mg, 33 mg-60 mg, 36 mg-38 mg, 36 mg-40 mg, 36 mg-42 mg, 36 mg-44 mg, 36 mg-46 mg, 36 mg-48 mg, 36 mg-50 mg, 36 mg-52 mg, 36 mg-54 mg, 36 mg-56 mg, 36 mg-58 mg, 36 mg-60 mg, 40 mg-42 mg, 40 mg-44 mg, 40 mg-46 mg, 40 mg-48 mg, 40 mg-50 mg, 40 mg-52 mg, 40 mg-54 mg, 40 mg-56 mg, 40 mg-58 mg, 40 mg-60 mg, 43 mg-46 mg, 43 mg-48 mg, 43 mg-50 mg, 43 mg-52 mg, 43 mg-54 mg, 43 mg-56 mg, 43 mg-58 mg, 42 mg-60 mg, 45 mg-48 mg, 45 mg-50 mg, 45 mg-52 mg, 45 mg-54 mg, 45 mg-56 mg, 45 mg-58 mg, 45 mg-60 mg, 48 mg-50 mg, 48 mg-52 mg, 48 mg-54 mg, 48 mg-56 mg, 48 mg-58 mg, 48 mg-60 mg, 50 mg-52 mg, 50 mg-54 mg, 50 mg-56 mg, 50 mg-58 mg, 50 mg-60 mg, 52 mg-54 mg, 52 mg-56 mg, 52 mg-58 mg, or 52 mg-60 mg. In some embodiments, a compound of Formula (I) dose is greater than about 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, or about 200 mg. In some embodiments, a compound of Formula (I) dose is about less than about 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, or about 200 mg.

In some embodiments, the treatment schedule includes administration of a compound of Formula (I) once every 1 week, 2 weeks, 3 weeks, 4 weeks, 5 weeks, 6 weeks, 7 weeks, or 8 weeks. In some embodiments, the treatment schedule includes administration of a compound of Formula (I) two times every 1 week, 2 weeks, 3 weeks, 4 weeks, 5 weeks, 6 weeks, 7 weeks, or 8 weeks. In some embodiments, the treatment schedule includes administration of a compound of Formula (I) once every 1 week in a treatment cycle of 1 week, 2 weeks, 3 weeks, 4 weeks, 5 weeks, 6 weeks, 7 weeks, or 8 weeks. In some embodiments, the treatment schedule includes administration of a compound of Formula (I) twice every 1 week in a treatment cycle of 1 week, 2 weeks, 3 weeks, 4 weeks, 5 weeks, 6 weeks, 7 weeks, or 8 weeks. In some embodiments, the treatment schedule includes the administration of a compound of Formula (I) on day 1, day 8, and day 15 of a 21-day treatment cycle.

The treatment cycle can be repeated as long as the regimen is clinically tolerated. In some embodiments, the treatment cycle for a compound of Formula (I) is repeated for n times, wherein n is an integer in the range of 2 to 30. In some embodiments, n is 2, 3, 4, 5, 6, 7, 8, 9, or 10. In some embodiments, a new treatment cycle can occur immediately after the completion of the previous treatment cycle. In some embodiments, a new treatment cycle can occur a period of time after the completion of the previous treatment cycle. In some embodiments, a new treatment cycle can occur after 1 week, 2 weeks, 3 weeks, 4 weeks, 5 weeks, 6 weeks, or 7 weeks after the completion of the previous treatment cycle.

### EXAMPLES

To further illustrate this disclosure, the following examples are included. The examples should not, of course, be construed as specifically limiting the disclosure. Variations of these examples within the scope of the claims are within the purview of one skilled in the art and are considered to fall within the scope of the disclosure as described, and claimed herein. The reader will recognize that the skilled artisan, armed with the present disclosure, and skill in the art is able to prepare and use the disclosure without exhaustive examples.

### Example 1

In the present study, plinabulin was characterized for its ability to inhibit anchorage independent growth and ex vivo colony formation of tumor cells in semi-solid medium. The compound was investigated in 87 out of 93 originally planned tumor xenografts representing all major cancer types using a 3D clonogenic assay in a 96-well format with colony count based on image analysis as read-out. By using this assay, the investigational compound was assessed for the ability to inhibit anchorage independent growth and ex vivo colony formation of tumor cells in semi-solid medium.

A master stock solution of plinabulin was prepared by dissolving the compound in DMSO at a concentration of 9.9 mM. Working stock solutions were prepared by diluting the master stock solution with DMSO to a concentration of 0.99 mM (330-fold the highest test concentration). Small aliquots of the master and working stock were stored at −20° C. On each day of an experiment, an aliquot of the working stock was used and stored at room temperature prior to and during treatment. All liquid handling steps were performed using a Tecan Freedom EVO 200 robotic platform. First, serial dilutions of the working stock solution were done in DMSO. The DMSO dilutions were then diluted 1:22 into cell culture medium (IMDM, supplemented with 20% (v/v) fetal calf serum, and 50 μg/ml gentamicin) in an intermediate dilution plate. Finally, 10 μl taken from the intermediate dilution plate were transferred to 140 μl/well of the final assay plate. Thus, the DMSO working stock was diluted 1:330, and the DMSO concentration in the assay was 0.3% v/v in all wells.

Plinabulin was investigated in 87 PDX models representing a number of cancer types. Tumors were passaged as subcutaneous xenografts in NMRI nu/nu mice. At a tumor volume of 400-1000 mm³ tumor-bearing mice were euthanized and tumors were collected under sterile conditions without delay according to relevant SOPs and the relevant animal welfare guidelines published by the FELASA and the GV-SOLAS. Tumors were mechanically disaggregated and subsequently incubated with an enzyme cocktail consisting of collagenase type IV (41 U/ml), DNase I (125 U/ml), hyaluronidase type III (100 U/ml), and dispase II (1 U/ml) in RPMI 1640 medium (Life Technologies) at 37° C. for 60-120 minutes. Cells were passed through sieves of 100 μm and 40 μm mesh size (Cell Strainer, BD Falcon™), and washed with RPMI 1640 medium (Biochrom). The percentage of viable cells was determined in a Neubauer-hemocytometer using trypan blue exclusion. Aliquots of the cells were frozen down and stored in a liquid nitrogen vapor phase. On each day of an experiment, a frozen aliquot of tumor cells was thawed and used for the preparation of assay plates.

The clonogenic assay was carried out in a 96 well plate format using ultra low attachment plates. For each test, cells were prepared as described above and assay plates were prepared as follows: each test well contained a layer of semi-solid medium with tumor cells (50 μl), and a second layer of medium supernatant with or without test compound (100 μl). The cell layer consisted of 2.5-12.5×10³ tumor cells per well, which were seeded on day 0 (d0) in 50 μl/well cell culture medium (IMDM, supplemented with 20% (v/v) fetal calf serum, 50 μg/ml gentamicin, and 0.4% (w/v) agar). After 24 h, the soft-agar layer was covered with 90 μl of the same culture medium without agar. Seven days after seeding 10 μl of test compound or control medium was added, and left on the cells for 24 h. Every 96 well plate included six DMSO-treated control wells and drug-treated wells in duplicate at 9 concentrations. After the incubation time of 24 h the supernatant (90 μl medium without agar +10 μl compound/control) was changed against drug free medium and incubated until the end of the study. Cultures were incubated at 37° C. and 7.5% CO₂ in a humidified atmosphere 13 days in total and monitored closely for colony growth using an inverted microscope. Within this period, ex vivo tumor growth led to the formation of colonies with a diameter of >50 μm (area >2000 μm²). At the time of maximum colony formation in the Max vehicle treated wells, vital colonies were stained for 48 h with a sterile aqueous solution of 2-(4-iodophenyl)-3-(4-nitrophenyl)-5-phenyltetrazolium chloride (INT, 1 mg/ml, 25 μl/well), and colony counts were performed with an automatic image analysis system (Bioreader 5000 V-alpha, BIO-SYS GmbH).

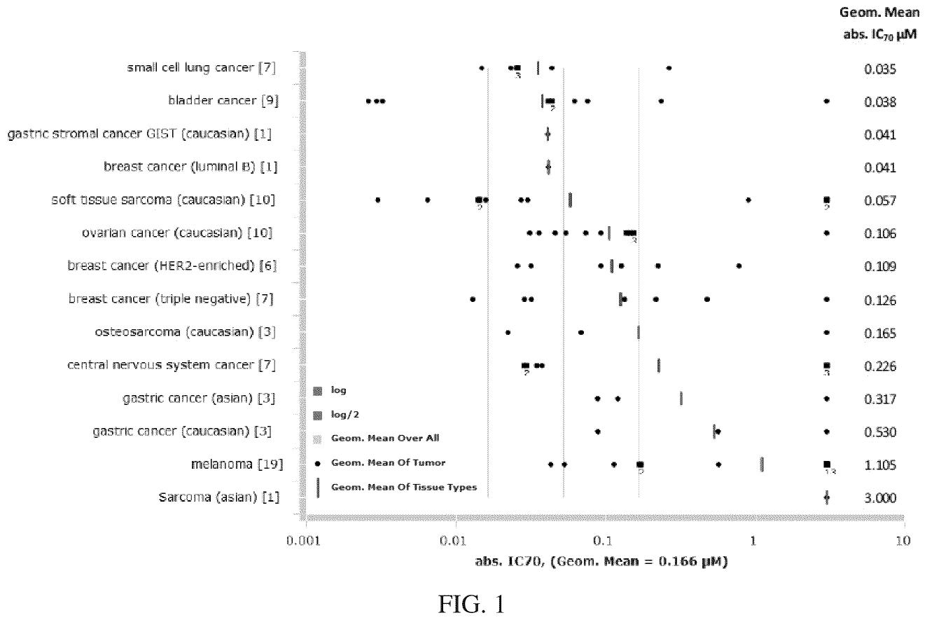

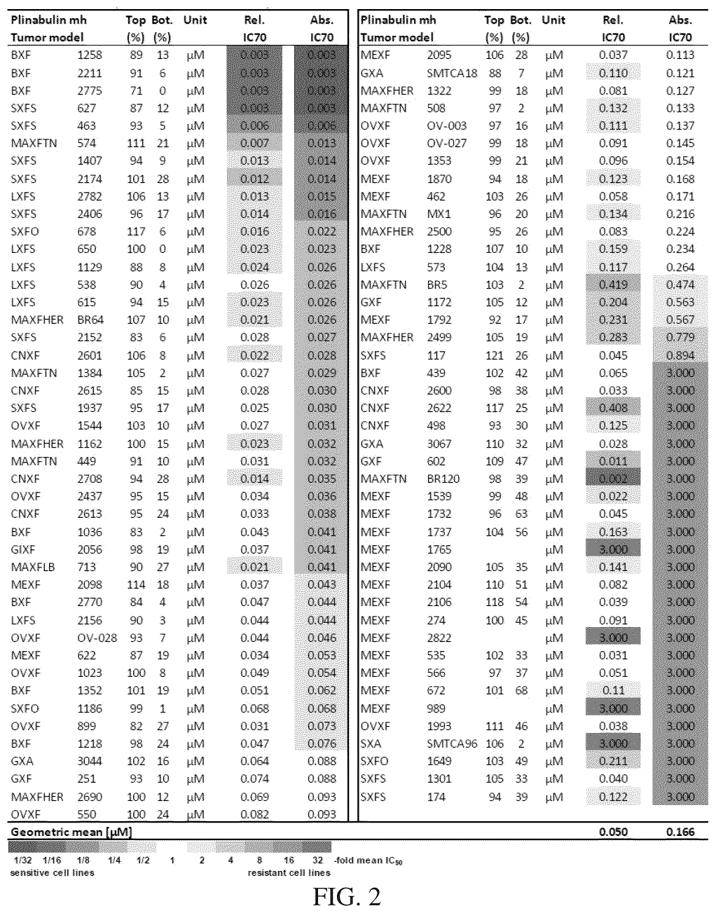

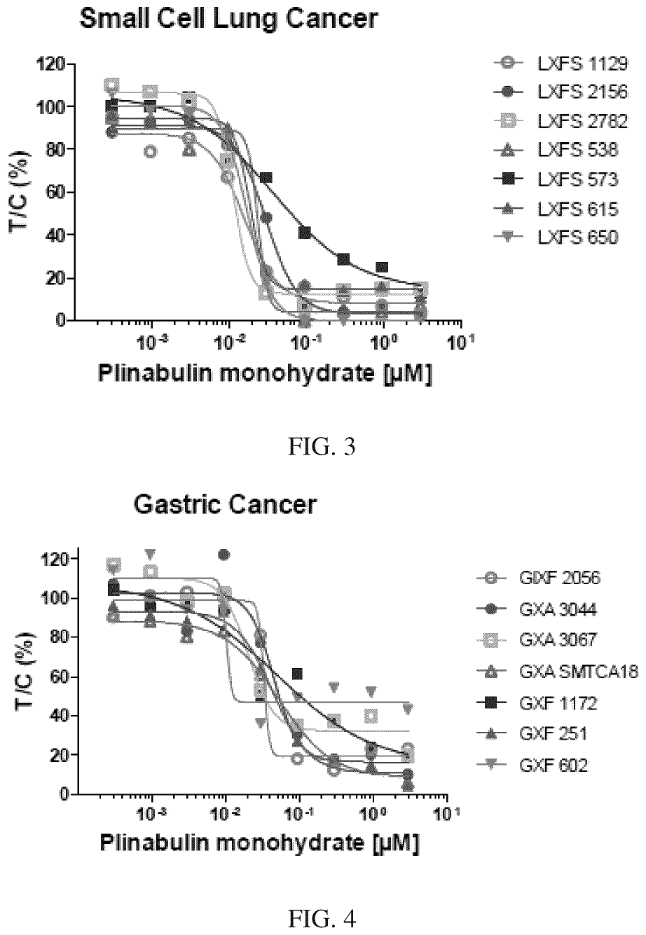

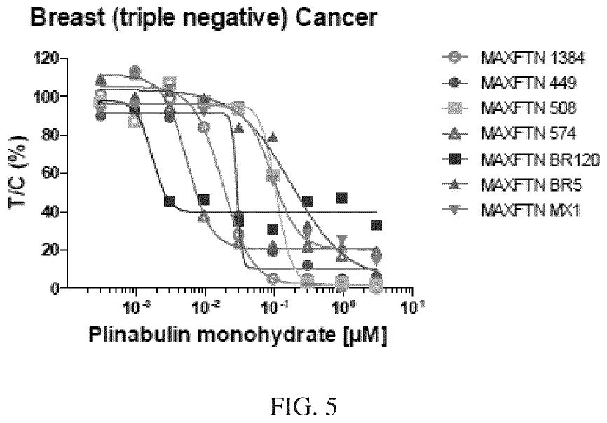

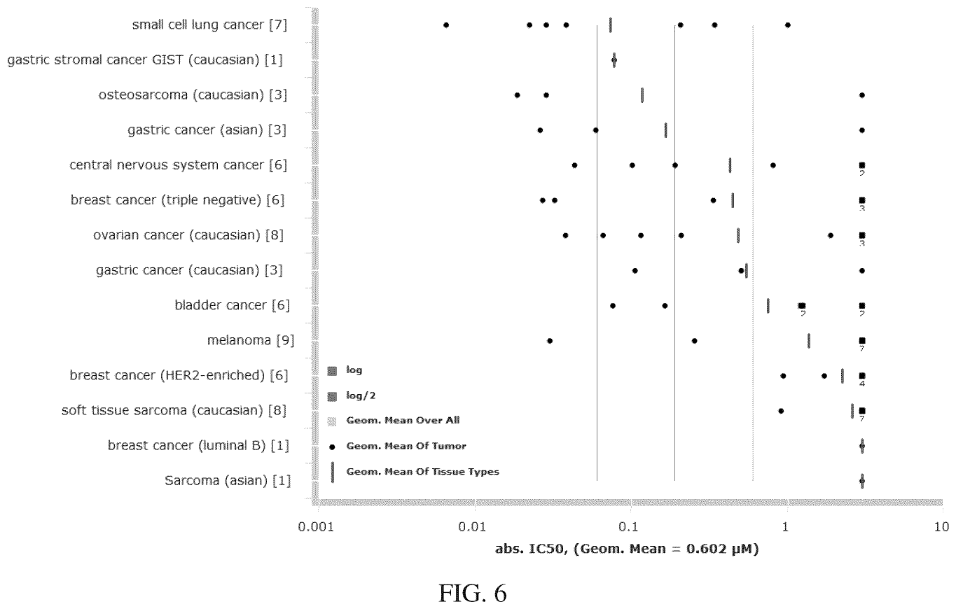

The ability of plinabulin to inhibit ex vivo colony formation of cells with the capability to grow in an anchorage-independent manner in semi-solid medium was examined in 87 PDX models of various histotypes using a 3D clonogenic assay. Results are summarized in FIG. 1 and a heatmap presentation of absolute and relative IC₇₀ values in FIG. 2. Concentration-effect curves for small cell lung cancer, gastric cancer, and triple negative breast cancer are shown in FIGS. 3-5.

Plinabulin inhibited tumor colony formation in a concentration-dependent manner in almost all cell lines resulting in sigmoidal concentration-effect curves as displayed in FIGS. 3-5, and a geometric mean absolute IC₇₀ value of 0.166 μM. Among all tumor models tested 40 out of 87 were sensitive towards plinabulin with a 6.8-fold difference of absolute IC₇₀ of sensitive models versus all models (Table 1).

TABLE 1

| Sensitive Tumors* | Based on Rel. IC₇₀ | Based on Abs. IC₇₀ |
|---|---|---|
| Number | 23/87 | 40/87 |
| Percentage | 26% | 46% |

Fold difference of IC₇₀ of sensitive tumors*

| | | |
|---|---|---|
| vs. all tumors | 4.6 | 6.8 |

*geometric mean of IC₅₀s of sensitive tumors relative to geometric mean of IC₇₀s of all models

The most sensitive histotypes based on geometric mean absolute IC₇₀ values were small cell lung cancer (geom. mean absolute IC₇₀=0.035 μM; n=7), bladder cancer (geom. mean absolute IC₇₀=0.038 μM; n=9), and soft tissue sarcoma (geom. mean absolute IC₇₀=0.057 μM; n=10).

Overall, the most responsive models were BXF 1258, BXF 2211, BXF 2775, and SXFS 627 with an absolute IC₇₀ of 3 nM and melanoma models were observed to be the most resistant ones with a geometric mean absolute IC₇₀ of 1.105 μM (n=19).

### Example 2

The present study investigated plinabulin for anticancer activity ex vivo in 68 out of 71 originally planned patient-derived xenograft (PDX) models representing major cancer types. Experiments were performed using a 3D clonogenic assay in a 96-well format with colony count based on image analysis as a read-out. By using this assay, the investigational compound was assessed for the ability to inhibit anchorage independent growth and ex vivo colony formation of tumor cells in semi-solid medium.

A master stock solution of plinabulin was prepared by dissolving the compound in DMSO at a concentration of 9.9 mM. Working stock solutions were prepared by diluting the master stock solution with DMSO to a concentration of 0.99 mM (330-fold the highest test concentration). Small aliquots of the master and working stock were stored at −20° C. On each day of an experiment, an aliquot of the working stock was used and stored at room temperature prior to and during treatment. All liquid handling steps were performed using a Tecan Freedom EVO 200 robotic platform. First, serial dilutions of the working stock solution were done in DMSO. The DMSO dilutions were then diluted 1:22 into the cell culture medium (IMDM, supplemented with 20% (v/v) fetal calf serum, and 50 μg/ml gentamicin) in an intermediate dilution plate. Finally, 10 μl taken from the intermediate dilution plate were transferred to 140 μl/well of the final assay plate. Thus, the DMSO working stock was diluted at 1:330, and the DMSO concentration in the assay was 0.3% v/v in all wells.

Plinabulin was investigated in 68 PDX models representing a number of cancer types. Tumors were passaged as subcutaneous xenografts in NMRI nu/nu mice. At a tumor volume of 400-1000 mm³ tumor-bearing mice were euthanized and tumors were collected under sterile conditions without delay according to relevant SOPs and the relevant animal welfare guidelines published by the FELASA and the GV-SOLAS. Tumors were mechanically disaggregated and subsequently incubated with an enzyme cocktail consisting of collagenase type IV (41 U/ml), DNase I (125 U/ml), hyaluronidase type III (100 U/ml), and dispase II (1 U/ml) in RPMI 1640 medium (Life Technologies) at 37° C. for 60-120 minutes. Cells were passed through sieves of 100 μm and 40 μm mesh size (Cell Strainer, BD Falcon™), and washed with RPMI 1640 medium (Biochrom). The percentage of viable cells was determined in a Neubauer-hemocytometer using trypan blue exclusion. Aliquots of the cells were frozen down and stored in a liquid nitrogen vapor phase. On each day of an experiment, a frozen aliquot of tumor cells was thawed and used for the preparation of assay plates.

The clonogenic assay was carried out in a 96 well plate format using ultra low attachment plates. For each test, cells were prepared as described above (Section 6.6) and assay plates were prepared as follows: each test well contained a layer of semi-solid medium with tumor cells (50 μl), and a second layer of medium supernatant with or without test compound (100 μl). The cell layer consisted of 2.5-12.5·10³ tumor cells per well, which were seeded on day 0 (d0) in 50 μl/well cell culture medium (IMDM, supplemented with 20% (v/v) fetal calf serum, 50 μg/ml gentamicin, and 0.4% (w/v) agar). After 24 h, the soft-agar layer was covered with 90 μl of the same culture medium without agar. Seven days after seeding 10 μl of test compound or control medium was added, and left on the cells for 24 h. Every 96 well plate included six DMSO-treated control wells and drug-treated wells in duplicate at 9 concentrations. After the incubation time of 24 h the supernatant (90 μl medium without agar +10 μl compound/control) was changed against drug free medium and incubated until the end of the study. Cultures were incubated at 37° C. and 7.5% CO₂ in a humidified atmosphere 13 days in total and monitored closely for colony growth using an inverted microscope. Within this period, ex vivo tumor growth led to the formation of colonies with a diameter of >50 μm (area >2000 μm²). At the time of maximum colony formation in the Max vehicle treated wells, vital colonies were stained for 48 h with a sterile aqueous solution of 2-(4-iodophenyl)-3-(4-nitrophenyl)-5-phenyltetrazolium chloride (INT, 1 mg/ml, 25 μl/well), and colony counts were performed with an automatic image analysis system (Bioreader 5000 V-alpha, BIO-SYS GmbH).

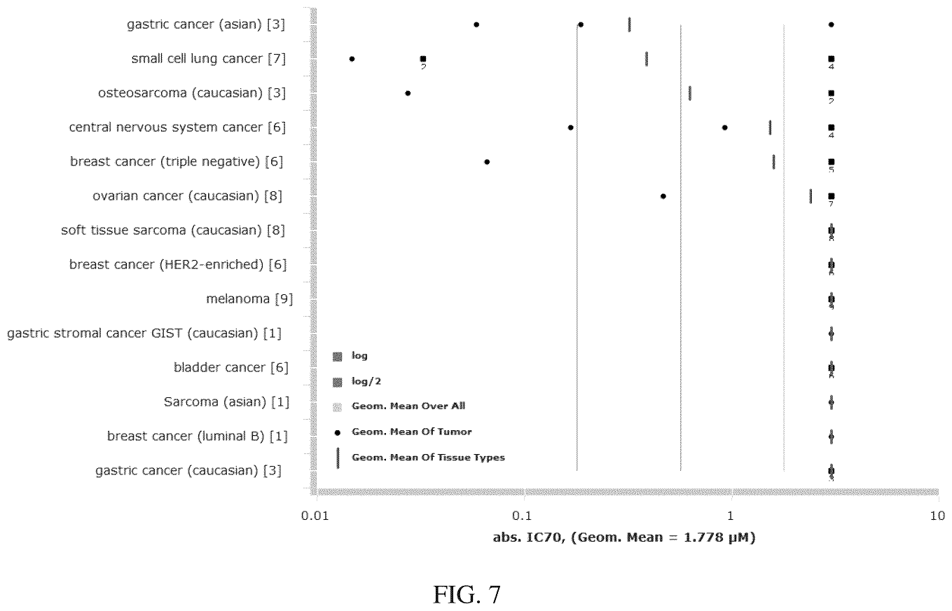

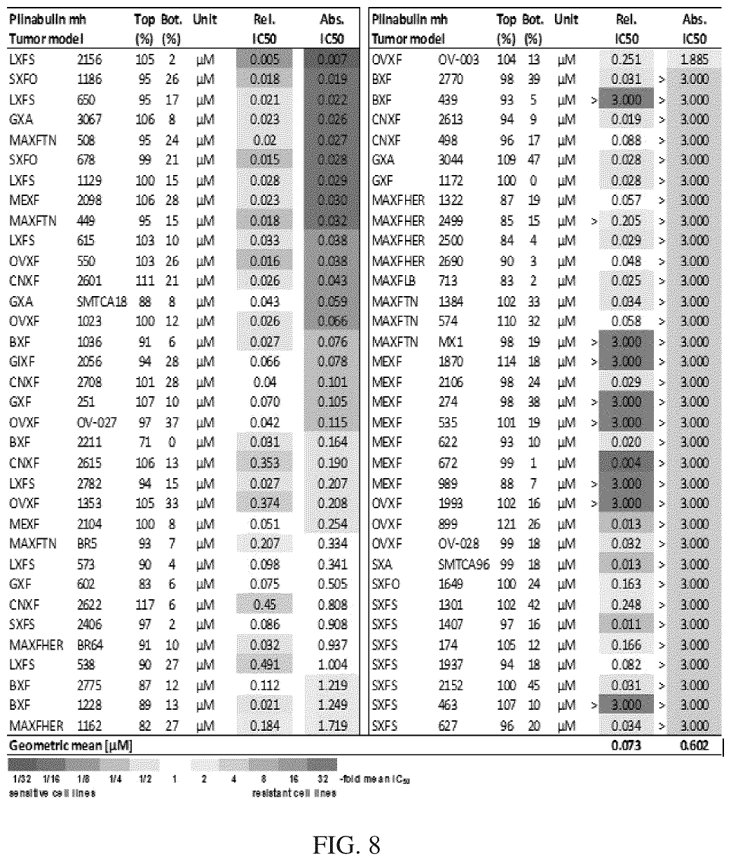

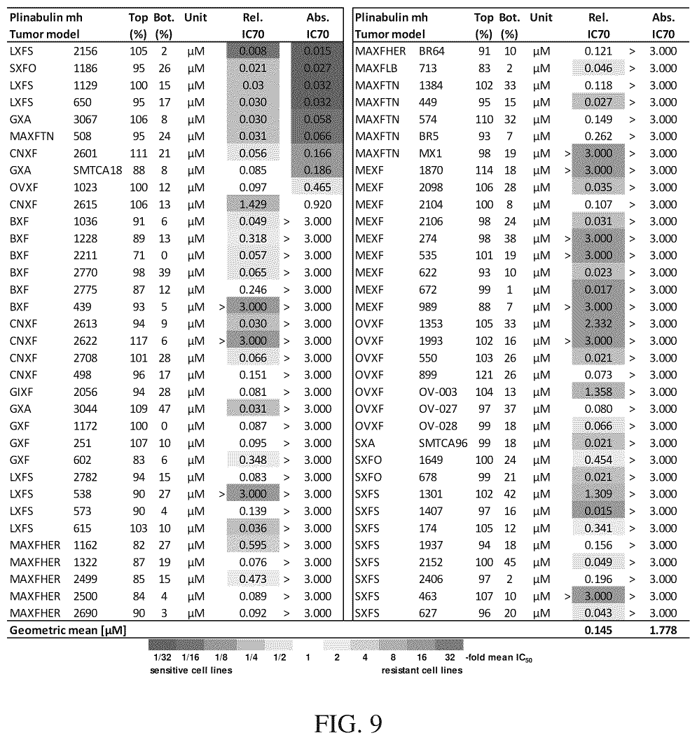

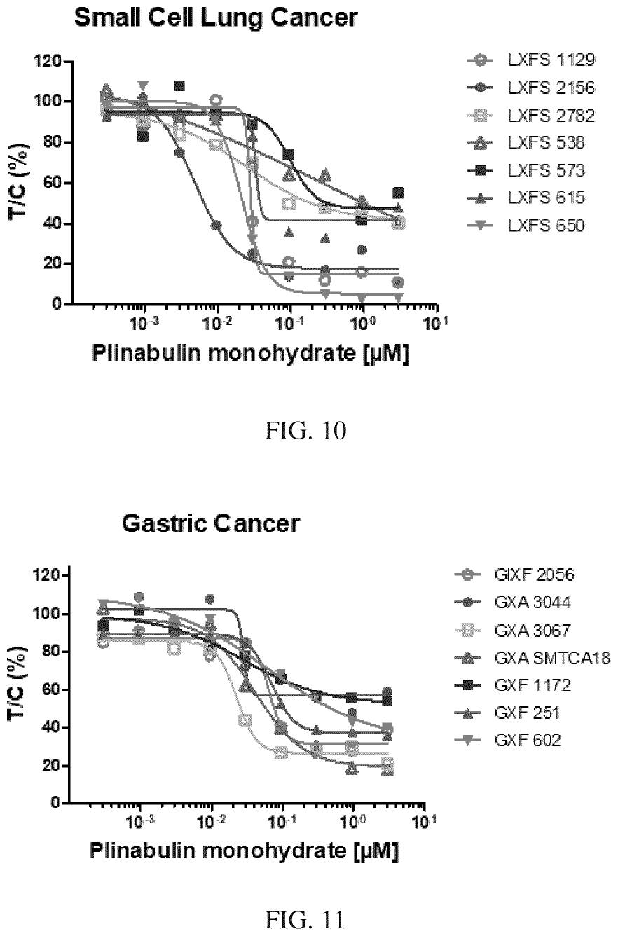

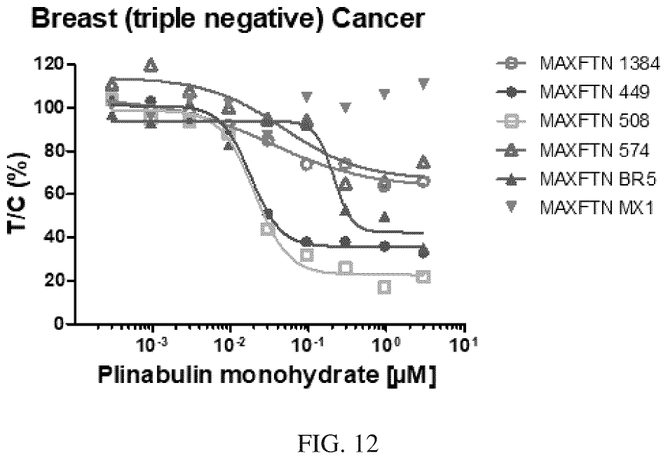

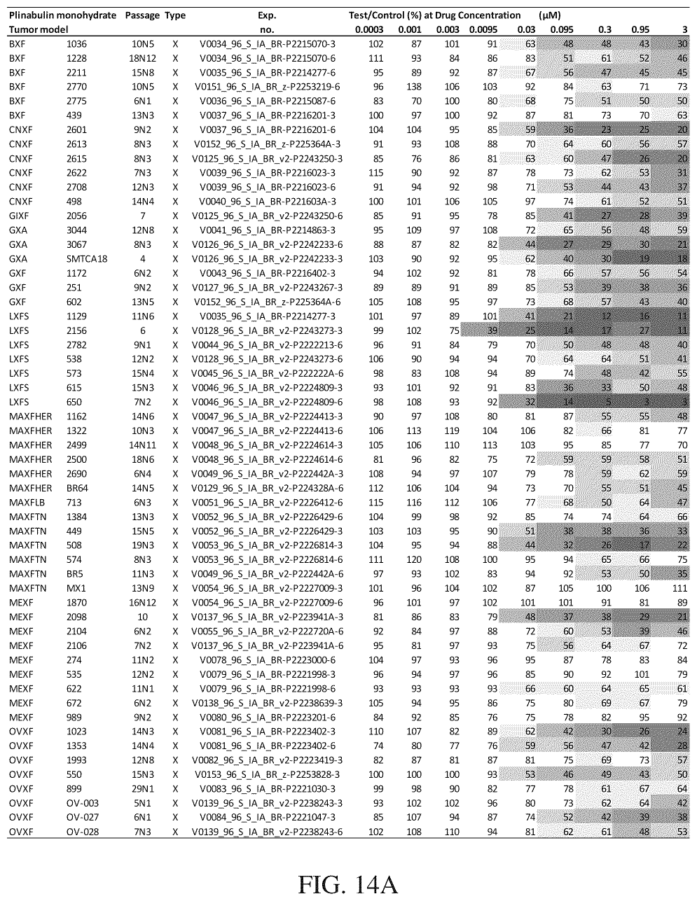

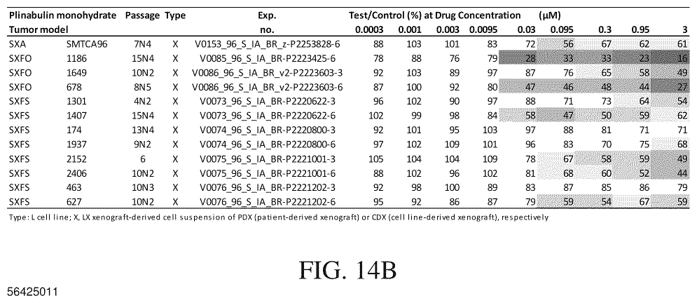

The ability of plinabulin to inhibit ex vivo colony formation of cells with the capability to grow in an anchorage-independent manner in a semi-solid medium was examined in 68 PDX models of various histotypes using a 3D clonogenic assay. Results are presented based on IC₅₀ and based on IC₇₀ as scatter plots (FIG. 6 and FIG. 7, respectively) and heatmaps of absolute and relative IC values (FIG. 8 and FIG. 9, respectively). Individual results, i.e. relative and absolute IC₅₀/IC₇₀ values (FIG. 13A and FIG. 13B), and concentration-response tables containing T/C values were determined (FIG. 14A and FIG. 14B). Concentration response curves for small cell lung cancer, gastric cancer, and triple negative breast cancer are shown in FIGS. 10-12, respectively

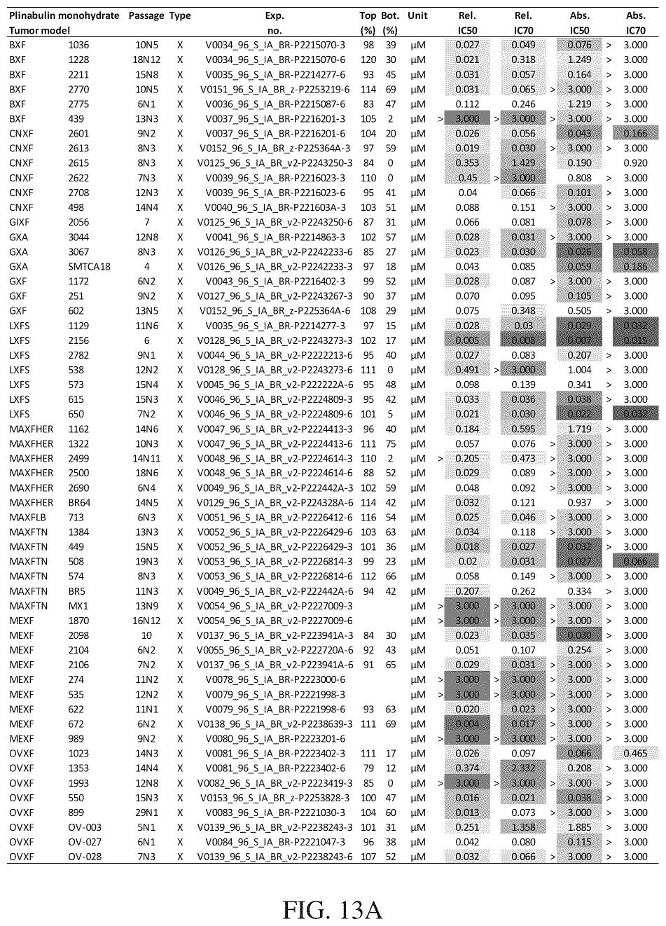

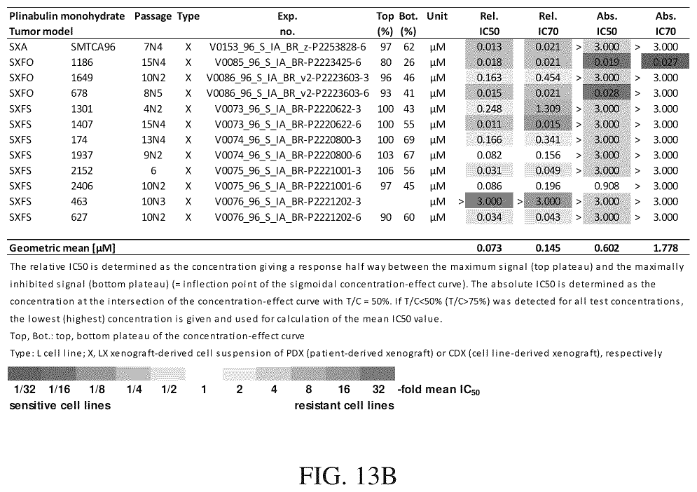

Plinabulin inhibited tumor colony formation in a concentration-dependent manner in almost all cell lines resulting in sigmoidal concentration-effect curves as displayed in FIG. 10, FIG. 11, and FIG. 12. Compared to the concentration-effect curves obtained in Example 1 in which cells were treated from day 2 to day 3, treatment from day 7 to day 8 in the present study led to higher bottom plateaus and increased IC₅₀ and IC₇₀ values. However, small cell lung cancer models were observed to be the most sensitive models in both studies of Example 1 and Example 2.

Plinabulin inhibited tumor colony formation with a geometric mean absolute IC₅₀ value of 0.6 μM (FIG. 6 and FIG. 9). Based on absolute IC₅₀ 24 out of 68 tumor models were sensitive toward plinabulin with a 10.7-fold difference of absolute IC₅₀ of sensitive versus all models (Table 2).

TABLE 2

| Sensitive Tumors* | Based on Rel. IC₅₀ | Based on Abs. IC₅₀ |
|---|---|---|
| Number | 34/68 | 24/68 |
| Percentage | 50% | 35% |

Fold difference of IC50 of sensitive tumors*

| | | |
|---|---|---|
| vs. all tumors | 3.4 | 10.7 |

*geometric mean of IC₅₀s of sensitive tumors relative to geometric mean of IC₅₀s of all models

The most sensitive histotypes based on geometric mean absolute IC₅₀ values (FIG. 6) were small cell lung cancer, (geom. mean abs. IC₅₀=0.074 μM, n=7), osteosarcoma (geom. mean abs. IC₅₀=0.117 μM, n=3), gastric cancer (Asian, geom. mean abs. IC₅₀=0.166 μM, n=3), and central nervous system cancer (geom. mean abs. IC₅₀=0.426 μM, n=6). Overall, the most responsive models based on abs. IC₅₀ (FIG. 8) were the small cell lung cancer models LXFS 2156 (abs. IC₅₀=0.007 μM) and LXFS 650 (abs. IC₅₀=0.022 μM), the osteosarcoma model SXFO 1186 (abs. IC₅₀=0.019 μM), and the (Asian) gastric cancer model GXA 3067 (abs. IC₅₀=0.026 μM). Melanoma models (geom. mean abs. IC₅₀=1.367 μM, n=9), Her2-enriched breast cancer models (geom. mean abs. IC₅₀=2.252 μM, n=6), and soft tissue sarcoma models (geom. mean abs. IC₅₀=2.584 μM, n=8) were observed to be the most resistant tumor types.

Plinabulin inhibited tumor colony formation with a geometric mean absolute IC₇₀ value of 1.78 μM (FIG. 7 and FIG. 9). Based on absolute IC₇₀, 9 out of 68 tumor models were sensitive towards plinabulin with a 27.1-fold difference of absolute IC₇₀ of sensitive versus all models (Table 3).

TABLE 3

| Sensitive Tumors* | Based on Rel. IC₇₀ | Based on Abs. IC₇₀ |
|---|---|---|
| Number | 27/68 | 9/68 |
| Percentage | 40% | 13% |

Fold difference of IC₇₀ of sensitive tumors*

| | | |
|---|---|---|
| vs. all tumors | 4.6 | 27.1 |

*geometric mean of IC₇₀s of sensitive tumors relative to geometric mean of IC₇₀s of all models

The most sensitive histotypes based on geometric mean absolute IC₇₀ values (FIG. 7) were gastric cancer (Asian, geom. mean abs. IC₇₀=0.319 μM, n=3), small cell lung cancer (geom. mean abs. IC₇₀=0.385 μM, n=7), osteosarcoma (geom. mean abs. IC₇₀=0.624 μM, n=3), and central nervous system cancer (geom. mean abs. IC₇₀=1.521 μM, n=6). Overall, the most responsive models based on abs. IC₇₀ (FIG. 9) were the small cell lung cancer models LXFS 2156 (abs. IC₇₀=0.015 μM), LXFS 1129 (abs. IC₇₀=0.032 μM) and LXFS 650 (abs. IC₇₀=0.032 μM), and the osteosarcoma model SXFO 1186 (abs. IC₇₀=0.027 μM). Triple negative breast cancer (MAXFTN) was also observed to be sensitive to plinabulin (see FIG. 7). Soft tissue sarcoma models (n=8), Her2-enriched breast cancer models (n=6), melanoma models (n=9), bladder cancer models (n=6), and gastric cancer models (Caucasian, n=3) were observed to be the most resistant tumor types with a geometric mean absolute IC₇₀ value>3 μM.

What is claimed is:

1. A method of treating a cancer in a subject in need thereof, the method comprising:
administering to the subject a compound of Formula (I) as a monotherapy, 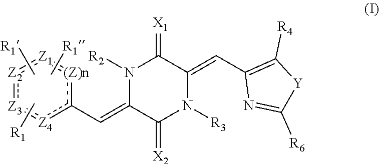
or a pharmaceutically acceptable salt thereof, wherein:
R₁, R₄, and R₆, are each separately selected from the group consisting of a hydrogen atom, a deuterium atom, a halogen atom, and saturated C₁-C₂₄ alkyl, unsaturated C₁-C₂₄ alkenyl, cycloalkyl, cycloalkenyl, alkoxy, cycloalkoxy, aryl, substituted aryl, heteroaryl, substituted heteroaryl, amino, substituted amino, nitro, azido, substituted nitro, phenyl, and substituted phenyl groups, hydroxy, carboxy, —CO—O—R₇, cyano, alkylthio, halogenated alkyl including polyhalogenated alkyl, halogenated carbonyl, and carbonyl —CH₂CO—R₇, wherein R₇ is selected from a hydrogen atom, a halogen atom, and saturated C₁-C₂₄ alkyl, unsaturated C₁-C₂₄ alkenyl, cycloalkyl, cycloalkenyl, alkoxy, cycloalkoxy, aryl, substituted aryl, heteroaryl, substituted heteroaryl, amino, substituted amino, nitro, azido, substituted nitro, phenyl, and substituted phenyl groups;
R₁′ and R₁″ are each independently selected from the group consisting of a hydrogen atom, a deuterium atom, a halogen atom, and saturated C₁-C₂₄ alkyl, unsaturated C₁-C₂₄ alkenyl, cycloalkyl, cycloalkenyl, alkoxy, cycloalkoxy, aryl, substituted aryl, heteroaryl, substituted heteroaryl, amino, substituted amino, nitro, azido, substituted nitro, phenyl, and substituted phenyl groups, hydroxy, carboxy, —CO—O—R₇, cyano, alkylthio, halogenated alkyl including polyhalogenated alkyl, halogenated carbonyl, and carbonyl —CH₂CO—R₇, wherein R₇ is selected from a hydrogen atom, a halogen atom, and saturated C₁-C₂₄ alkyl, unsaturated C₁-C₂₄ alkenyl, cycloalkyl, cycloalkenyl, alkoxy, cycloalkoxy, aryl, substituted aryl, heteroaryl, substituted heteroaryl, amino, substituted amino, nitro, azido, substituted nitro, phenyl, and substituted phenyl groups;
R, R₁′ and R₁″ are either covalently bound to one another or are not covalently bound to one another;
R₂, R₃, and R₅ are each separately selected from the group consisting of a hydrogen atom, a deuterium atom, a halogen atom, and saturated C₁-C₁₂ alkyl, unsaturated C₁-C₁₂ alkenyl, acyl, cycloalkyl, alkoxy, cycloalkoxy, aryl, substituted aryl, heteroaryl, substituted heteroaryl, amino, substituted amino, nitro, and substituted nitro groups, sulfonyl and substituted sulfonyl groups;
m is an integer equal to zero, one or two;
X₁ and X₂ are separately selected from the group consisting of an oxygen atom, a nitrogen atom and a sulfur atom, and
Y is selected from the group consisting of a NR₅, an oxygen atom, a sulfur atom, a oxidized sulfur atom, a methylene group and a substituted methylene group;
Z, for each separate n, if non-zero, and Z₁, Z₂, Z₃ and Z₄ are each separately selected from a carbon atom, a sulfur atom, a nitrogen atom or an oxygen atom; and
the dashed bonds may be either single or double bonds,
wherein the cancer is selected from gastric cancer, small cell lung cancer, osteosarcoma, bladder cancer, and triple negative breast cancer.

2. The method of claim 1, wherein the cancer is gastric cancer.

3. The method of claim 1, wherein the cancer is small cell lung cancer.

4. The method of claim 1, wherein the cancer is triple negative breast cancer.

5. The method of any one of claims 1 to 4, wherein the compound of Formula (I) is administered at a dose from about 5 mg/m² to 150 mg/m².

6. The method of any one of claims 1 to 4, wherein the compound of Formula (I) is administered at a dose from about 10 mg/m² to 50 mg/m².

7. The method of any one of claims 1 to 4, wherein the compound of Formula (I) is administered at a dose from about 20 mg/m² to 30 mg/m².

8. The method of any one of claims 1 to 7, wherein the compound of Formula (I) is administered at a dose of about 20 mg/m².

9. The method of any one of claims 1 to 7, wherein the compound of Formula (I) is administered at a dose of about 30 mg/m².

10. The method of any one of claims 1 to 6, wherein the compound of Formula (I) is administered at a dose of about 40 mg/m².

11. The method of any one of claims 1 to 10, wherein the compound of Formula (I) is administered on day 1 of a 14 day dosing cycle.

12. The method of any one of claims 1 to 10, wherein the compound of Formula (I) is administered on day 1 of a 21 day dosing cycle.

13. The method of any one of claims 1 to 12, wherein the compound of Formula (I) is selected from plinabulin, (3Z,6Z)-3-(phenyl-2,3,4,5,6-d₅)-methylene-6-((5-(tert-butyl)-1H-imidazol-4-yl)methylene)piperazine-2,5-dione; (3Z,6Z)-3-(phenyl-2,3,4,5,6-d₅)-methylene-d-6-((5-(tert-butyl)-1H-imidazol-4-yl)methylene)piperazine-2,5-dione; (3Z,6Z)-3-(phenylmethylene-d)-6-((5-(tert-butyl)-1H-imidazol-4-yl)methylene-d)piperazine-2,5-dione; (3Z,6Z)-3-(phenyl-2,3,4,5,6-d₅)-methylene-6-((5-(tert-butyl)-1H-imidazol-4-yl)methylene-d)piperazine-2,5-dione; (3Z,6Z)-3-(phenylmethylene)-6-((5-(tert-butyl)-1H-imidazol-4-yl)methylene-d)piperazine-2,5-dione; (3Z,6Z)-3-(phenyl-2,3,4,5,6-d₅)-methylene-d-6-((5-(tert-butyl)-1H-imidazol-4-yl)methylene-d)piperazine-2,5-dione; (3Z,6Z)-3-(4-Fluoro-(phenyl-2,3,5,6-d₄))-methylene-6-((5-(tert-butyl)-1H-imidazol-4-yl)methylene)piperazine-2,5-dione; (3Z,6Z)-3-(4-Fluoro-(phenyl-2,3,5,6-d₄))-methylene-6-((5-(tert-butyl)-1H-imidazol-4-yl)methylene-d)piperazine-2,5-dione; (3Z,6Z)-3-(3-fluorobenzylidene)-6-((5-(tert-butyl)-1H-imidazol-4-yl)methylene-d)piperazine-2,5-dione; (3Z,6Z)-3-(3-benzoylbenzylidene)-6-((5-(tert-butyl)-1H-imidazol-4-yl)methylene-d)piperazine-2,5-dione; (3Z,6Z)-3-(3-(4-fluorobenzoyl)benzylidene)-6-((5-(tert-butyl)-1H-imidazol-4-yl)methylene-d)piperazine-2,5-dione; (3Z,6Z)-3-(3-(4-methoxybenzoyl)benzylidene)-6-((5-(tert-butyl)-1H-imidazol-4-yl)methylene-d)piperazine-2,5-dione; (3Z,6Z)-3-(3-methoxybenzylidene)-6-((5-(tert-butyl)-1H-imidazol-4-yl)methylene-d)piperazine-2,5-dione; (3Z,6Z)-3-(3-(trifluoromethyenzydene)-6-((5-(tert-butyl)-1H-imidazol-4-yl)methylene-d)piperazine-2,5-dione; and a pharmaceutically acceptable salt thereof.

14. The method of any one of claims 1 to 13, wherein the compound of Formula (I) is plinabulin or a pharmaceutically acceptable salt thereof.

15. The method of any one of claims 1 to 14, wherein the cancer comprises a tumor and a mass of the tumor is reduced from about 50% to about 100%.

16. The method of claim 15, wherein the tumor mass is reduced from about 50% to about 70%.

17. A method of halting or reversing a progressive cancer in a subject, the method comprising:
administering a compound of Formula (I) as a monotherapy, 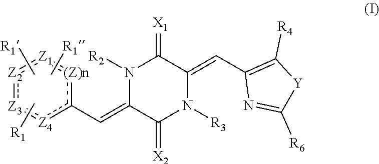
or a pharmaceutically acceptable salt thereof, wherein:
R₁, R₄, and R₆, are each separately selected from the group consisting of a hydrogen atom, a deuterium atom, a halogen atom, and saturated C₁-C₂₄ alkyl, unsaturated C₁-C₂₄ alkenyl, cycloalkyl, cycloalkenyl, alkoxy, cycloalkoxy, aryl, substituted aryl, heteroaryl, substituted heteroaryl, amino, substituted amino, nitro, azido, substituted nitro, phenyl, and substituted phenyl groups, hydroxy, carboxy, —CO—O—R₇, cyano, alkylthio, halogenated alkyl including polyhalogenated alkyl, halogenated carbonyl, and carbonyl —CH₂CO—R₇, wherein R₇ is selected from a hydrogen atom, a halogen atom, and saturated C₁-C₂₄ alkyl, unsaturated C₁-C₂₄ alkenyl, cycloalkyl, cycloalkenyl, alkoxy, cycloalkoxy, aryl, substituted aryl, heteroaryl, substituted heteroaryl, amino, substituted amino, nitro, azido, substituted nitro, phenyl, and substituted phenyl groups;
R₁′ and R₁″ are each independently selected from the group consisting of a hydrogen atom, a deuterium atom, a halogen atom, and saturated C₁-C₂₄ alkyl, unsaturated C₁-C₂₄ alkenyl, cycloalkyl, cycloalkenyl, alkoxy, cycloalkoxy, aryl, substituted aryl, heteroaryl, substituted heteroaryl, amino, substituted amino, nitro, azido, substituted nitro, phenyl, and substituted phenyl groups, hydroxy, carboxy, —CO—O—R₇, cyano, alkylthio, halogenated alkyl including polyhalogenated alkyl, halogenated carbonyl, and carbonyl —CH₂CO—R₇, wherein R₇ is selected from a hydrogen atom, a halogen atom, and saturated C₁-C₂₄ alkyl, unsaturated C₁-C₂₄ alkenyl, cycloalkyl, cycloalkenyl, alkoxy, cycloalkoxy, aryl, substituted aryl, heteroaryl, substituted heteroaryl, amino, substituted amino, nitro, azido, substituted nitro, phenyl, and substituted phenyl groups;
R, R₁′ and R₁″ are either covalently bound to one another or are not covalently bound to one another;
R₂, R₃, and R₅ are each separately selected from the group consisting of a hydrogen atom, a deuterium atom, a halogen atom, and saturated C₁-C₁₂ alkyl, unsaturated C₁-C₁₂ alkenyl, acyl, cycloalkyl, alkoxy, cycloalkoxy, aryl, substituted aryl, heteroaryl, substituted heteroaryl, amino, substituted amino, nitro, and substituted nitro groups, sulfonyl and substituted sulfonyl groups;
m is an integer equal to zero, one or two;
X₁ and X₂ are separately selected from the group consisting of an oxygen atom, a nitrogen atom and a sulfur atom, and
Y is selected from the group consisting NRs, an oxygen atom, a sulfur atom, a oxidized sulfur atom, a methylene group and a substituted methylene group;
Z, for each separate n, if non-zero, and Z₁, Z₂, Z₃ and Z₄ are each separately selected from a carbon atom, a sulfur atom, a nitrogen atom or an oxygen atom;
the dashed bonds may be either single or double bonds,
wherein the cancer is selected from gastric cancer, small cell lung cancer, osteosarcoma, bladder cancer, and triple negative breast cancer.

18. The method of claim 17, wherein the cancer is gastric cancer.

19. The method of claim 17, wherein the cancer is small cell lung cancer.

20. The method of claim 17, wherein the cancer is triple negative breast cancer.

21. The method of any one of claims 17 to 20, wherein the compound of Formula (I) is administered at a dose from about 5 mg/m² to 150 mg/m².

22. The method of any one of claims 17 to 20, wherein the compound of Formula (I) is administered at a dose from about 10 mg/m² to 50 mg/m².

23. The method of any one of claims 17 to 20, wherein the compound of Formula (I) is administered at a dose from about 20 mg/m² to 30 mg/m².

24. The method of any one of claims 17 to 20, wherein the compound of Formula (I) is administered at a dose of about 20 mg/m².

25. The method of any one of claims 17 to 20, wherein the compound of Formula (I) is administered at a dose of about 30 mg/m².

26. The method of any one of claims 17 to 20, wherein the compound of Formula (I) is administered at a dose of about 40 mg/m².

27. The method of any one of claims 17 to 26, wherein the compound of Formula (I) is administered on day 1 of a 14 day dosing cycle.

28. The method of any one of claims 17 to 26, wherein the compound of Formula (I) is administered on day 1 of a 21 day dosing cycle.

29. The method of any one of claims 17 to 28, wherein the compound of Formula (I) is plinabulin or a pharmaceutically acceptable salt thereof.

30. The method of any one of claims 17 to 29, wherein the cancer comprises a tumor and a mass of the tumor is reduced from about 50% to about 100%.

31. The method of claim 30, wherein the tumor mass is reduced from about 50% to about 70%.

32. A method of inhibiting proliferation of a cancer cell, the method comprising:
contacting the cancer cell with an effective amount of plinabulin and no other chemotherapeutic agent,
wherein the cancer cell is from a cancer selected from gastric cancer, small cell lung cancer, osteosarcoma, bladder cancer, and triple negative breast cancer.

33. The method of claim 32, wherein the cancer cell is gastric cancer.

34. The method of claim 32, wherein the cancer cell is small cell lung cancer.

35. The method of claim 32, wherein the cancer cell is triple negative breast cancer.

36. The method of any one of claims 32 to 35, wherein the compound of Formula (I) is administered at a dose from about 5 mg/m² to 150 mg/m².

37. The method of any one of claims 32 to 35, wherein the compound of Formula (I) is administered at a dose from about 10 mg/m² to 50 mg/m².

38. The method of any one of claims 32 to 35, wherein the compound of Formula (I) is administered at a dose from about 20 mg/m² to 30 mg/m².

39. The method of any one of claims 32 to 35, wherein the compound of Formula (I) is administered at a dose of about 20 mg/m².

40. The method of any one of claims 32 to 35, wherein the compound of Formula (I) is administered at a dose of about 30 mg/m².

41. The method of any one of claims 32 to 35, wherein the compound of Formula (I) is administered at a dose of about 40 mg/m².

42. The method of any one of claims 32 to 41, wherein the compound of Formula (I) is administered on day 1 of a 14 day dosing cycle.

43. The method of any one of claims 32 to 41, wherein the compound of Formula (I) is administered on day 1 of a 21 day dosing cycle.

44. The method of any one of claims 32 to 43, wherein the compound of Formula (I) is plinabulin or a pharmaceutically acceptable salt thereof.

45. The method of any one of claims 32 to 44, wherein the cancer cell comprises a tumor cell and a mass of the tumor cell is reduced from about 50% to about 100%.

46. The method of claim 45, wherein the tumor cell mass is reduced from about 50% to about 70%.

47. A method of inducing apoptosis in a cancer cell, the method comprising:
contacting the cancer cell with an effective amount of plinabulin and no other chemotherapeutic agent,
wherein the cancer is selected from gastric cancer, small cell lung cancer, osteosarcoma, bladder cancer, and triple negative breast cancer.

48. The method of claim 47, wherein the compound of Formula (I) is administered at a dose from about 10 mg/m² to 50 mg/m².

49. The method of claim 47 or 48, wherein the cancer cell comprises a tumor cell and a mass of the tumor cell is reduced from about 50% to about 100%.

50. The method of any one of claims 47 to 49, wherein the compound of Formula (I) is administered on day 1 of a 21 day dosing cycle.
